(19) United States
(12) Patent Application Publication
(10) Pub. No.: US 2017/0068792 A1
(43) Pub. Date: Mar. 9, 2017

(54) SYSTEM AND METHOD FOR MEDICAL DEVICE SECURITY, DATA TRACKING AND OUTCOMES ANALYSIS

(71) Applicant: Bruce REINER, Berlin, MD (US)

(72) Inventor: Bruce REINER, Berlin, MD (US)

(21) Appl. No.: 15/257,208

(22) Filed: Sep. 6, 2016

Related U.S. Application Data
(60) Provisional application No. 62/213,855, filed on Sep. 3, 2015.

(51) Int. Cl. G06F 19/00; A61B 5/00; A61B 10/02; A61B 6/03; A61B 17/12; A61B 17/88; A61B 5/055; A61F 2/01; A61F 2/02; A61N 1/362; A61F 2/82; A61B 5/06; A61B 6/00

(52) U.S. Cl. CPC G06F 19/3406; A61B 5/066; A61B 5/0022; A61B 5/743; A61B 10/0283; A61B 6/032; A61B 17/1214; A61B 6/501; A61B 5/055; A61B 5/0042; A61F 2/01; A61B 6/481; A61F 2/022; A61N 1/362; A61F 2/82; A61B 17/8822; A61F 2250/0096; A61B 2560/0271; A61B 2560/0276

(57) ABSTRACT

The present invention creates an objective methodology of quantitative accountability for medical device manufacturers, vendors, clinical providers, patients, and payers. In one embodiment, the standardized data received and stored in the medical device database can in turn be used for a variety of applications related to decision support (e.g., medical device selection), education and training (e.g., procedural performance), cost efficacy, evidence based medicine and best practice guidelines, personalized medicine, and comparative performance/safety analytics.

### CROSS-REFERENCE TO THE RELATED APPLICATIONS

The present invention claims priority from U.S. Provisional Patent Application No. 62/213,855 filed Sep. 3, 2015, the contents of which are herein incorporated by reference in their entirety.

### BACKGROUND OF THE INVENTION

1. Field of the Invention

The present invention relates to a system and method for medical device security, data tracking and outcomes analysis.

2. Description of the Related Art

Medical device safety is a risk management process which should (but currently does not) encompass the complete life span of the medical device, from conception to disposal. Optimum safety and performance requires multi-party compliance, cooperation, and communication in order to ensure that all relevant medical device data is properly documented, stored, analyzed, and reported in accordance with community wide standards and best practice guidelines. The multiple parties involved in this collective process include device manufacturers, importers, vendors, regulatory and enforcement agencies, institutional healthcare providers, individual clinical providers, patients, payers, researchers, and medico-legal professionals.

The major phases in the lifespan of a medical device include the following:

1. Conception and development

2. Device manufacture

3. Packaging and labeling

4. Advertising

5. Sale

6. Use

7. Disposal

The first three phases are largely the responsibility of the device manufacturer, who is tasked with creation of the medical device and ensuring that all device safety and performance standards are met and maintained in the individual steps of device development, manufacture, packaging, and labeling. The manufacturer is tasked with pre-market surveillance and testing to ensure that existing performance and safety standards are met in order to achieve regulatory approval from authorized governmental agencies (e.g., Food and Drug Administration (FDA)).

The fourth and fifth phases are primarily the responsibility of the device vendor which serves as the interface between the product and end-user. The vendor is responsible for ensuring that device advertising and sales are in keeping with regulatory requirements, while also ensuring that after-sales service (e.g., device training, education, equipment maintenance, quality control) is provided to ensure device safety and performance standards are adequately maintained.

The sixth and seventh phases are largely the collective responsibilities of device end-users, which include the institutional service and individual clinical providers. In addition to ensuring that all involved end-users are properly certified, educated, and credentialed in medical device use, these healthcare providers are also responsibility for ensuring that all aspects related to device selection, utilization, and clinical usage are commensurate with community standards and best practice guidelines.

In current practice, medical device data collection is largely restricted to pre-market analysis, which is encompassed in the FDA approval process which ensures that medical device quality and safety standards are achieved commensurate with its intended clinical use. Post-market data analysis is intrinsically lacking and dependent upon voluntary data reporting by manufacturers and end-users, which in large part restrict reporting to device related “significant” adverse events. Since reporting standards are lax, many device-related clinical events go unreported and as a result are never fully realized by regulatory agencies, which are tasked with ensuring ongoing post-market medical device safety.

Along with the paucity of post-market data tied to medical device quality and safety, another looming problem is readily apparent yet largely continues. This is the high and unnecessary economic costs associated with overutilization, fraudulent billing, device counterfeiting and repackaging, and increased medico-legal liability associated with medical devices. Overutilization is often the result of lucrative financial incentives to physician users which can take a variety of forms including (but not limited to) lucrative consulting agreements, agreements, creation of Physician Owned Distributorships (PODs), kickbacks and royalty payments, unrestricted grants, and payment for educational conferences and travel. Fraudulent billing represents illicit and/or excessive billing for services which were not (or improperly) performed. Device counterfeiting and repackaging occurs when disreputable parties create illicit products and/or repackage products after use for the purpose of reselling these products as new and unused. Increased medico-legal costs are largely borne from the lack of prospective data collection, analysis, and intervention related to medical device quality and safety, resulting in unintended and unnecessary morbidity and mortality, and eventually leading to costly class action lawsuits.

In response to these well documented clinical and economic deficiencies, the FDA has recently issued a rule requiring medical device labels to include unique identifiers which can be incorporated into electronic healthcare records. While this requirement represents a good starting point for tracking medical devices, it does not address the myriad of fundamental concerns and lack of data related to post-market device usage. In order to accurately and reliably perform post-market surveillance and analysis of medical devices each individual step, participant, and technology must be accounted for in the collective process of medical device usage, beginning with device selection and ending with device disposal. A number of data requirements are essential to ensure the derived quality, safety, and economic analytics are reproducible and accurate. The data must be collected prospectively, reported in an automated fashion, exist in a standardized format, and be verifiable and secure.

While existing rules and regulations require manufacturers and users to report significant adverse clinical events related to medical device usage, documentation and reporting of medical device safety and performance data is inherently lacking in conventional medical practice.

In order to address the existing deficiencies in medical device safety and performance a standardized method of step-wise data collection is needed which provides objective and reproducible accountability for all phases of the medical device life span as well as all participating parties.

### SUMMARY OF THE INVENTION

The present invention relates to a system and method for medical device security, data tracking and outcomes analysis, including supporting technologies which enable the creation, recording, storage, communication, analysis, and reporting of standardized quality and safety metrics throughout the medical device life span. This data can in turn be used for clinical decision support, creation of best practice guidelines (i.e., Evidence-Based Medicine (EBM)), automated communication networks and analytics, and customizable healthcare delivery (i.e., Personalized Medicine).

The present invention addresses the myriad of existing deficiencies in medical device quality, safety, and economics by creating a referenceable database comprised of objective and standardized metrics. In order to facilitate the creation of these metrics, a number of supporting medical device technologies are created which provide for the collection of real-time medical device data, which in turn can lead to proactive intervention in the event of device malfunction and/or adverse clinical outcome. This in effect leads to the creation of “smart” medical devices which can facilitate real-time data analysis and communication between the involved parties; with the ultimate goals of simultaneously improving medical device quality, safety, and economics.

In one embodiment, a computer-implemented method of providing ensuring medical device functionality, includes: providing a medical device for internal use within a patient during a medical procedure, the medical device having sensors or biomarkers disposed therein for providing data on the medical device and the patient; confirming the medical device data integrity and device functionality by receiving data from the medical device into a database of a computer system and performing an analysis using a processor of the computer system; and confirming, using the processor, a position of the medical device within the patient using an imaging device or a positional analysis of positional data from the data from the medical device; wherein predetermined changes in the position of the medical device are monitored for indication of an adverse event.

In one embodiment, the medical device includes electronic tags which contain medical device information that can be scanned by a scanner and saved in the database.

In one embodiment, when a data outlier is detected during the analysis, performing a data reconciliation process using the processor, to identify erroneous, insufficient or abnormal data relative to the best practice guidelines.

In one embodiment, when the data outlier is determined as abnormal, using the processor, generating an escalation pathway to analyze a cause and a severity of the data, in order to determine whether an intervention should be performed.

In one embodiment, the method further includes: generating an alert by electronic means when a contraindication is identified during the analysis by the processor.

In one embodiment, the sensors or biomarkers provide continuous data after completion of the medical procedure.

In one embodiment, an appropriateness of the medical procedure and the medical device are included in the analysis.

In one embodiment, a standardized model for training, education, and proof of clinical competency with respect to medical devices is determined during the analysis.

In one embodiment, a GPS in the medical device provides anatomic real-time position and continuous data.

In one embodiment, the analysis includes clinical outcomes analysis and analysis of providers to generate customized medical device decision-making relative to peer and community wide standards.

In one embodiment, the method further includes: continuously monitoring quality and safety metrics of at least patients, providers, and the medical devices.

In one embodiment, the method compares data on the position of the medical device within the patient with comparable patients and medical devices using the processor.

In one embodiment, the method generates best practices guidelines using the processor, based on the compared data, for use of the medical device with patients.

Thus, has been outlined, some features consistent with the present invention in order that the detailed description thereof that follows may be better understood, and in order that the present contribution to the art may be better appreciated. There are, of course, additional features consistent with the present invention that will be described below and which will form the subject matter of the claims appended hereto.

In this respect, before explaining at least one embodiment consistent with the present invention in detail, it is to be understood that the invention is not limited in its application to the details of construction and to the arrangements of the components set forth in the following description or illustrated in the drawings. Methods and apparatuses consistent with the present invention are capable of other embodiments and of being practiced and carried out in various ways. Also, it is to be understood that the phraseology and terminology employed herein, as well as the abstract included below, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the methods and apparatuses consistent with the present invention.

### BRIEF DESCRIPTION OF THE DRAWINGS

FIG. 1 is a schematic diagram which shows the overall components of the apparatus of the present invention, according to one embodiment consistent with the present invention.

FIG. 2 is a schematic drawing of a partial cutaway side view and a cross-sectional view of a catheter with embedded sensors, according to one embodiment consistent with the present invention.

FIGS. 3A-3C are a flowchart which shows the principal steps in the method of usage of the medical device, according to one embodiment consistent with the present invention.

FIG. 4 is a schematic drawing of a partial cutaway side view and a cross-sectional view of a nanobot biosensor, according to one embodiment consistent with the present invention.

FIGS. 5A and 5B are schematic drawings showing partial cutaway side views of a receiving catheter and delivery catheter, with biosensors and reservoirs, according to one embodiment consistent with the present invention.

FIGS. 6A and 6B show a system with needle injection apparatus, pump, reservoir and diagnostic sensors, according to one embodiment consistent with the present invention.

FIG. 7 is a schematic drawing of an implanted surgical medical device, according to one embodiment consistent with the present invention.

FIG. 8 is a schematic drawing of a medical device specific sensor roadmap, according to one embodiment consistent with the present invention.

FIG. 9 is a schematic drawing of various medical devices implanted in the human body, according to one embodiment consistent with the present invention.

### DESCRIPTION OF THE INVENTION

The present invention relates to a system and method for medical device security, data tracking and outcomes analysis, including supporting technologies which enable the creation, recording, storage, communication, analysis, and reporting of standardized quality and safety metrics throughout the medical device life span. This data can in turn be used for clinical decision support, creation of best practice guidelines (i.e., Evidence-Based Medicine (EBM)), automated communication networks and analytics, and customizable healthcare delivery (i.e., Personalized Medicine). The present invention encompasses a wide array of clinical, technical, and economic applications, which collectively are aimed at improving clinical outcomes, medical device security, patient safety, and cost efficacy.

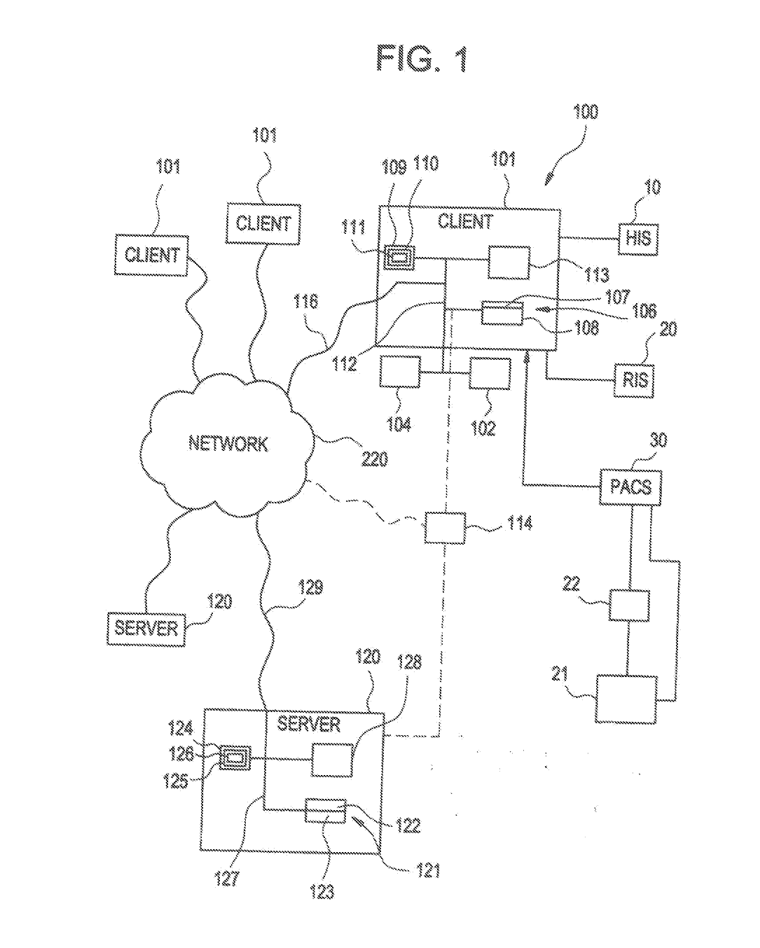

According to one embodiment of the invention as illustrated in FIG. 1, medical applications may be implemented using the system 100. The system 100 is designed to interface with existing information systems such as a Hospital Information System (HIS) 10, a Radiology Information System (RIS) 20, a radiographic device 21, and/or other information systems, a Picture Archiving and Communication System (PACS) 30, and/or other systems. The system 100 may be designed to conform with the relevant standards, such as the Digital Imaging and Communications in Medicine (DICOM) standard, DICOM Structured Reporting (SR) standard, and/or the Radiological Society of North America's Integrating the Healthcare Enterprise (IHE) initiative, among other standards.

According to one embodiment, bi-directional communication between the system 100 of the present invention and the information systems, such as the HIS 10, RIS 20, Quality Assurance (QA) sensor device 22, and PACS 30, etc., may be enabled to allow the system 100 to retrieve and/or provide information from/to these systems. According to one embodiment of the invention, bi-directional communication between the system 100 of the present invention and the information systems allows the system 100 to update information that is stored on the information systems. According to one embodiment of the invention, bi-directional communication between the system 100 of the present invention and the information systems allows the system 100 to generate desired reports and/or other information.

The system 100 of the present invention includes a client computer 101, such as a personal computer (PC), which may or may not be interfaced or integrated with the PACS 30. The client computer 101 may include an imaging display device 102 that is capable of providing high resolution digital images in 2-D or 3-D, for example. According to one embodiment of the invention, the client computer 101 may be a mobile terminal if the image resolution is sufficiently high. Mobile terminals may include mobile computing devices, a mobile data organizer (PDA), tablet, smart phone, or other mobile terminals that are operated by the user accessing the program 110 remotely.

According to one embodiment of the invention, an input device 104 or other selection device, may be provided to select hot clickable icons, selection buttons, and/or other selectors that may be displayed in a user interface using a menu, a dialog box, a roll-down window, or other user interface. The user interface may be displayed on the client computer 101. According to one embodiment of the invention, users may input commands to a user interface through a programmable stylus, keyboard, mouse, speech processing device, laser pointer, touch screen, or other input device 104.

According to one embodiment of the invention, the input or other selection device 104 may be implemented by a dedicated piece of hardware or its functions may be executed by code instructions that are executed on the client processor 106. For example, the input or other selection device 104 may be implemented using the imaging display device 102 to display the selection window with a stylus or keyboard for entering a selection.

According to another embodiment of the invention, symbols and/or icons may be entered and/or selected using an input device 104, such as a multi-functional programmable stylus. The multi-functional programmable stylus may be used to draw symbols onto the image and may be used to accomplish other tasks that are intrinsic to the image display, navigation, interpretation, and reporting processes. The multi-functional programmable stylus may provide superior functionality compared to traditional computer keyboard or mouse input devices. According to one embodiment of the invention, the multi-functional programmable stylus also may provide superior functionality within the PACS and Electronic Medical Report (EMR).

According to one embodiment of the invention, the client computer 101 may include a processor 106 that provides client data processing. According to one embodiment of the invention, the processor 106 may include a central processing unit (CPU) 107, a parallel processor, an input/output (I/O) interface 108, a memory 109 with a program 110 having a data structure 111, and/or other components. According to one embodiment of the invention, the components all may be connected by a bus 112. Further, the client computer 101 may include the input device 104, the image display device 102, and one or more secondary storage devices 113. According to one embodiment of the invention, the bus 112 may be internal to the client computer 101 and may include an adapter that enables interfacing with a keyboard or other input device 104. Alternatively, the bus 112 may be located external to the client computer 101.

According to one embodiment of the invention, the image display device 102 may be a high resolution touch screen computer monitor. According to one embodiment of the invention, the image display device 102 may clearly, easily and accurately display images, such as x-rays, and/or other images. Alternatively, the image display device 102 may be implemented using other touch sensitive devices including tablet personal computers, pocket personal computers, plasma screens, among other touch sensitive devices. The touch sensitive devices may include a pressure sensitive screen that is responsive to input from the input device 104, such as a stylus, that may be used to write/draw directly onto the image display device 102.

According to another embodiment of the invention, high resolution lenses may be used as a graphical display to provide end users with the ability to review images. According to another embodiment of the invention, the high resolution lenses may provide graphical display without imposing physical constraints of an external computer.

According to another embodiment, the invention may be implemented by an application that resides on the client computer 101, wherein the client application may be written to run on existing computer operating systems. Users may interact with the application through a graphical user interface. The client application may be ported to other personal computer (PC) software, personal digital assistants (PDAs), cell phones, and/or any other digital device that includes a graphical user interface and appropriate storage capability.

According to one embodiment of the invention, the processor 106 may be internal or external to the client computer 101. According to one embodiment of the invention, the processor 106 may execute a program 110 that is configured to perform predetermined operations. According to one embodiment of the invention, the processor 106 may access the memory 109 in which may be stored at least one sequence of code instructions that may include the program 110 and the data structure 111 for performing predetermined operations. The memory 109 and the program 110 may be located within the client computer 101 or external thereto.

While the system of the present invention may be described as performing certain functions, one of ordinary skill in the art will readily understand that the program 110 may perform the function rather than the entity of the system itself.

According to one embodiment of the invention, the program 110 that runs the system 100 may include separate programs 110 having code that performs desired operations. According to one embodiment of the invention, the program 110 that runs the system 100 may include a plurality of modules that perform sub-operations of an operation, or may be part of a single module of a larger program 110 that provides the operation.

According to one embodiment of the invention, the processor 106 may be adapted to access and/or execute a plurality of programs 110 that correspond to a plurality of operations. Operations rendered by the program 110 may include, for example, supporting the user interface, providing communication capabilities, performing data mining functions, performing e-mail operations, and/or performing other operations.

According to one embodiment of the invention, the data structure 111 may include a plurality of entries. According to one embodiment of the invention, each entry may include at least a first storage area, or header, that stores the databases or libraries of the image files, for example.

According to one embodiment of the invention, the storage device 113 may store at least one data file, such as image files, text files, data files, audio files, video files, among other file types. According to one embodiment of the invention, the data storage device 113 may include a database, such as a centralized database and/or a distributed database that are connected via a network. According to one embodiment of the invention, the databases may be computer searchable databases. According to one embodiment of the invention, the databases may be relational databases. The data storage device 113 may be coupled to the server 120 and/or the client computer 101, either directly or indirectly through a communication network, such as a LAN, WAN, and/or other networks. The data storage device 113 may be an internal storage device. According to one embodiment of the invention, the system 100 may include an external storage device 114. According to one embodiment of the invention, data may be received via a network and directly processed.

According to one embodiment of the invention, the client computer 101 may be coupled to other client computers 101 or servers 120. According to one embodiment of the invention, the client computer 101 may access administration systems, billing systems and/or other systems, via a communication link 116. According to one embodiment of the invention, the communication link 116 may include a wired and/or wireless communication link, a switched circuit communication link, or may include a network of data processing devices such as a LAN, WAN, the Internet, or combinations thereof. According to one embodiment of the invention, the communication link 116 may couple e-mail systems, fax systems, telephone systems, wireless communications systems such as pagers and cell phones, wireless PDA's and other communication systems.

According to one embodiment of the invention, the communication link 116 may be an adapter unit that is capable of executing various communication protocols in order to establish and maintain communication with the server 120, for example. According to one embodiment of the invention, the communication link 116 may be implemented using a specialized piece of hardware or may be implemented using a general CPU that executes instructions from program 110. According to one embodiment of the invention, the communication link 116 may be at least partially included in the processor 106 that executes instructions from program 110.

According to one embodiment of the invention, if the server 120 is provided in a centralized environment, the server 120 may include a processor 121 having a CPU 122 or parallel processor, which may be a server data processing device and an I/O interface 123. Alternatively, a distributed CPU 122 may be provided that includes a plurality of individual processors 121, which may be located on one or more machines.

According to one embodiment of the invention, the processor 121 may be a general data processing unit and may include a data processing unit with large resources (i.e., high processing capabilities and a large memory for storing large amounts of data).

According to one embodiment of the invention, the server 120 also may include a memory 124 having a program 125 that includes a data structure 126, wherein the memory 124 and the associated components all may be connected through bus 127. If the server 120 is implemented by a distributed system, the bus 127 or similar connection line may be implemented using external connections. The server processor 121 may have access to a storage device 128 for storing preferably large numbers of programs 110 for providing various operations to the users.

According to one embodiment of the invention, the data structure 126 may include a plurality of entries, wherein the entries include at least a first storage area that stores image files. Alternatively, the data structure 126 may include entries that are associated with other stored information as one of ordinary skill in the art would appreciate.

According to one embodiment of the invention, the server 120 may include a single unit or may include a distributed system having a plurality of servers 120 or data processing units. The server(s) 120 may be shared by multiple users in direct or indirect connection to each other. The server(s) 120 may be coupled to a communication link 129 that is preferably adapted to communicate with a plurality of client computers 101.

According to one embodiment, the present invention may be implemented using software applications that reside in a client and/or server environment. According to another embodiment, the present invention may be implemented using software applications that reside in a distributed system over a computerized network and across a number of client computer systems. Thus, in the present invention, a particular operation may be performed either at the client computer 101, the server 120, or both.

According to one embodiment of the invention, in a client-server environment, at least one client and at least one server are each coupled to a network 220, such as a Local Area Network (LAN), Wide Area Network (WAN), and/or the Internet, over a communication link 116, 129. Further, even though the systems corresponding to the HIS 10, the RIS 20, the QA sensor 21, and the PACS 30 (if separate) are shown as directly coupled to the client computer 101, it is known that these systems may be indirectly coupled to the client over a LAN, WAN, the Internet, and/or other network via communication links. According to one embodiment of the invention, users may access the various information sources through secure and/or non-secure internet connectivity. Thus, operations consistent with the present invention may be carried out at the client computer 101, at the server 120, or both. The server 120, if used, may be accessible by the client computer 101 over the Internet, for example, using a browser application or other interface.

According to one embodiment of the invention, the client computer 101 may enable communications via a wireless service connection. The server 120 may include communications with network/security features, via a wireless server, which connects to, for example, voice recognition. According to one embodiment, user interfaces may be provided that support several interfaces including display screens, voice recognition systems, speakers, microphones, input buttons, and/or other interfaces. According to one embodiment of the invention, select functions may be implemented through the client computer 101 by positioning the input device 104 over selected icons. According to another embodiment of the invention, select functions may be implemented through the client computer 101 using a voice recognition system to enable hands-free operation. One of ordinary skill in the art will recognize that other user interfaces may be provided.

According to another embodiment of the invention, the client computer 101 may be a basic system and the server 120 may include all of the components that are necessary to support the software platform. Further, the present client-server system may be arranged such that the client computer 101 may operate independently of the server 120, but the server 120 may be optionally connected. In the former situation, additional modules may be connected to the client computer 101. In another embodiment consistent with the present invention, the client computer 101 and server 120 may be disposed in one system, rather being separated into two systems.

Although the above physical architecture has been described as client-side or server-side components, one of ordinary skill in the art will appreciate that the components of the physical architecture may be located in either client or server, or in a distributed environment.

Further, although the above-described features and processing operations may be realized by dedicated hardware, or may be realized as programs having code instructions that are executed on data processing units, it is further possible that parts of the above sequence of operations may be carried out in hardware, whereas other of the above processing operations may be carried out using software.

The underlying technology allows for replication to various other sites. Each new site may maintain communication with its neighbors so that in the event of a catastrophic failure, one or more servers 120 may continue to keep the applications running, and allow the system to load-balance the application geographically as required.

Further, although aspects of one implementation of the invention are described as being stored in memory, one of ordinary skill in the art will appreciate that all or part of the invention may be stored on or read from other computer-readable media, such as secondary storage devices, like hard disks, floppy disks, CD-ROM, or other forms of ROM or RAM either currently known or later developed. Further, although specific components of the system have been described, one skilled in the art will appreciate that the system suitable for use with the methods and systems of the present invention may contain additional or different components.

The present invention creates an objective methodology of quantitative accountability for medical device manufacturers, vendors, clinical providers, patients, and payers. In one embodiment, the standardized data received by the program 110 and stored in the medical device database 113, 114 can in turn be used by the program 110 for a variety of applications related to decision support (e.g., medical device selection), education and training (e.g., procedural performance), cost efficacy, evidence based medicine and best practice guidelines, personalized medicine, and comparative performance/safety analytics.

A number of these applications, embodiments, and their advantages are summarized below, and more detailed descriptions of program 110 operation follow.

### SUMMARY OF APPLICATIONS AND ADVANTAGES OF THE PRESENT INVENTION

1. Creation of a standardized method for ensuring medical device safety and security, with the program 110 creating electronic tags which are printed by a printer and attached to the outer packaging and intrinsic device. The data stored in the database 113, 114 and recorded within these electronic tags, from inputs received at a client computer 110, include the identity and location of the manufacturer, date/time device was verified and packaged, the specific attributes of the device, and identity of person/s responsible for quality control.

2. Standardized method of recording data into the database 113, 114 related to opening of device packaging and usage of the device. The electronic tags attached to the packaging and device, which are read by a scanning device into the database 113, 114, would serve as a dual method for documenting the date/time of device use along with recording of data related to the identities of the involved parties (i.e., institution, participating healthcare professionals, and patient of record) therein.

3. The specific data recorded by a scanner or inputted into a client computer 100, at the time of medical device unpacking and usage include the following:

a. Institutional data: Name, location, facility type, Historical Usage of Device, Profile Score.

b. Professional staff data: Names of involved staff, Occupation, Credentials, Educational information (including CME), Clinical Performance data, Historical usage of Device and/or Procedure to be performed, Provider Profile Scores.

c. Patient data: Name, Clinical history, Medical Diagnoses, Allergies, Laboratory and Imaging data, Prior Surgical and Procedural data (including complications), Profile Score (discussed below).

d. Device data: Date and Time of Use, Clinically Approved Indications (FDA, CMS, Professional Societal), Performance and Safety Ratings, Device Profile Score.

4. Automated recording of data into centralized database 113, 114 (using devices, such as scanners, with wireless transmission) with internal data analysis by the processor 106 which runs the program 110, for detection of data outliers. If data outlier is identified by the program 110, an automated data reconciliation process is initiated by the program 110 to identify whether the data recorded in the database 113, 114 was erroneous, insufficient, or truly abnormal relative to established norms and guidelines.

5. When a data outlier is verified to be abnormal (relative to established norms) by the program 110, an automated escalation pathway is triggered by the program 110 to analyze the cause and severity of the data outlier, with the possibility of intervention (when a compromised clinical outcome is of concern) (i.e., by notifying a physician by electronic means, such as fax, text, email, etc.).

6. When a contraindication is identified by the program 110, when cross-referencing the input data with the central database 113, 114 (e.g., high risk of complication for the device being used when correlated with the individual patient's or providers profile), an automated alert (i.e., by electronic means, such as fax, email, text, etc.) will be sent by the program 110 which requires acknowledgement and formal response by all involved parties. This also becomes an integral component of the informed consent. The program 110 will provide alternative options in accordance with database analysis by the program 110 of comparable patients and providers; such as an alternative device with a higher safety profile, alternative healthcare provider (i.e., physician) with a higher safety and clinical performance profile for the procedure and diagnosis being treated.

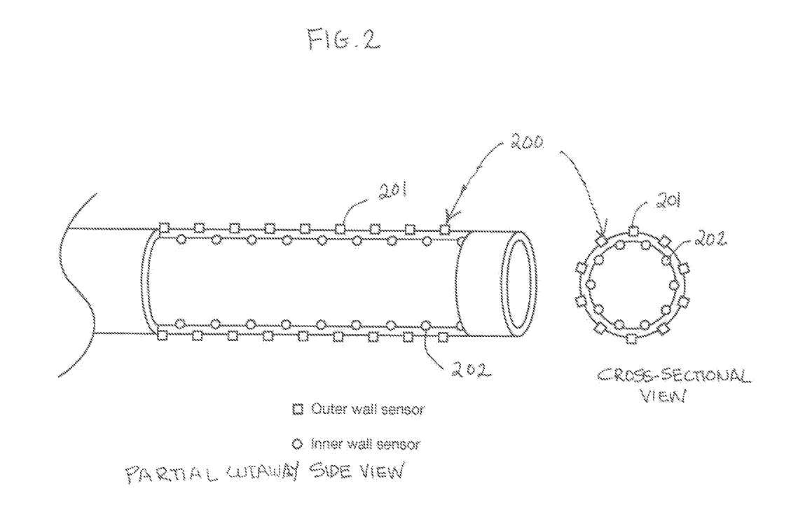

7. The medical device used on a patient may also contain embedded biosensors/biomarkers 21 which can be useful for continuous data analysis after completion of a medical procedure (see FIG. 2, for biosensor 200). As an example, if an SG catheter is inserted into a patient, an embedded biosensor 200 can continuously record pulmonary artery pressure measurements, and the program 110 can send an automated alert (by electronic means) to the desired receiver, when the pressure that is recorded in the database 113, 114, changes beyond a predefined threshold.

Another example may include a cardiac pacemaker (see FIG. 9) which records cardiac rate and rhythm into the database 113, 114, and where the program 110 sends an alert by electronic means when the baseline rate and rhythm is altered. These automated alerts can be transmitted by the program 110 using wireless transmission to the authorized providers, in accordance with a predefined notification/escalation pathway. The provider is required to respond to the data transmission and submit a follow up response.

Other medical devices include an artificial pancreas, abdominal pancreas, and artery stents, as shown in FIG. 9.

8. One application is coordinated billing and compliance with intended use. Standardized data within the database 113, 114 can lead to the creation of a standardized medical device referenceable database 113, 114 by the program 110. In addition to a myriad of clinical applications, the program 110 can also use the data to automate billing and reimbursement related to medical device usage. After registration of the medical device, provider, and patient in the database 113, 114, a computer-based device utilization and authorization can be performed by the program 110 which analyzes the appropriateness of the planned procedure and selected device, in accordance with established best practice guidelines. After authorization is performed, the clinical provider can proceed with the procedure and upon successful completion, a procedure and device billing can be automatically generated by the program 110, and payment initiated. In the event that the proposed procedure and/or medical device are not deemed to be appropriate by the program 110 (i.e., for that individual's medical condition), or requires modification for optimal clinical care, a reconciliation process will be automatically invoked by the program 110 which allows for direct input and communication between the clinical provider and third party payer. The ability to create a standardized and referenceable medical device database 113, 114 using the program 110 of the present invention, creates the opportunity for this automated billing in accordance with best practice guidelines.

9. An important application is mandatory training, education, and clinical proficiency. The program 110 of the present invention has the ability to create a standardized model for credentialing and proof of clinical competency with regards to performance of medical procedures related to different types of medical devices. In current practice, individual institutional providers create credentialing and clinical competency programs which may or may not be based upon medical societal standards. One particular challenge is when different types of medical specialists are performing the same or similar procedures (e.g., pulmonologist and radiologist performing lung biopsy, interventional radiologist and vascular surgeon inserting an aortic stent graft—see FIG. 9). Since each medical specialty society has its own methods and requirements for demonstrating clinical competency, this creates confusion and inconsistency in establishing credentialing and competency standards, even within the same institution.

In one embodiment, the present invention provides a single all-inclusive database 113, 114, and a program 110 which records, tracks, and analyzes safety, quality, and performance data referable to medical devices and the procedures associated with them. The fact that this data is standardized allows for meta-analysis by the program 110, and comingling of data from a large pool of institutional and individual providers, which provides an objective and unbiased mechanism for establishing best practice guidelines, community wide standards, and proficiency requirements specific to individual medical devices and associated procedures. In addition, the prospective and continuous nature of data collection allows for longitudinal performance monitoring and analysis by the program 110. In the event that a provider's quality and/or safety analytics are below that of their peers, the program 110 can provide customized feedback as to the deficiency of concern along with targeted educational and training materials along with the option for mentoring by volunteer “high performance” providers. In extreme or repetitive cases of poor performance quality/safety metrics, standardized guidelines can be established by the program 110 for remedial educational requirements, probation, or loss of clinical privileges. This collective process of standardized clinical proficiency and education/training requirements on a medical device/procedure specific basis is a unique and important feature of the invention, which is further supported by the ability of the program 110 analytics to continuously analyze and provider user and context-specific feedback.

10. Another application is GPS anatomic real-time localization and updates. A number of supporting technologies of the present invention provide for continuous update on medical device localization in vivo (i.e., within the human body). These localization technologies are not only used during the performance of the initial procedure (e.g., device insertion), but also continue throughout the lifetime of the medical device. Subtle changes in device positioning are a constant occurrence and if monitored can serve as an early warning sign for potentially adverse events (e.g., slippage of orthopedic surgical screws in the spine). The additional benefit of the program 110 continuously tracking this data over large number of patients and medical devices provides a mechanism for determining the threshold at which device locational changes may be of clinical significance and when intervention is required. In current practice, assessment of device location and positional change is cursory in nature, which results in significant pathology and positional change before providers and patients become aware. The present invention's technology, in effect, provides early warning signs of device positional change, along with determination of the clinical impact of these positional changes.

11. One application is for outcomes analysis and provider/patient specific performance assessment. The ability to automate the recording, by the program 110 into the database 113, 114, of standardized and objective data throughout the lifetime of the medical device (i.e., continuous data collection) provides a critical ability to continuously monitor device performance and how it relates to clinical outcomes. In addition, data specific to each individual provider and patient provides a method for user-specific analysis, which can be used to customize medical device decision making (i.e., personalized medicine) and provider performance analysis relative to peer and community wide standards (i.e., evidence-based medicine).

12. One application relates to quality assurance (QA) and quality control (QC) reporting and analysis. The data continuously collected in the database 113, 114 and analyzed by the program 110, can be used to continuously monitor quality and safety metrics of both the involved players (e.g., providers, patients, administrators) and the involved technologies (i.e., medical devices and their components (e.g. sensors). The ability of the program 110 to standardize methods for medical device quality control are a unique feature of the invention and provides important (and currently unavailable) data for determining device longevity and clinical utility of medical devices over prolonged periods of time. This ability of the program 110 to perform standardized QC monitoring and analysis is particularly important for medical devices which have an expected long lifespan (e.g., surgical implants, vascular grafts).

Thus, at the core of the present invention is the medical device database 113, 114 which is responsible for data collection, storage, analysis, reporting and communication. A wide array of program 110 features and functionality are included in the invention, which include (but are not limited to) the following:

Features of the Program and Universal Medical Device Database of the Present Invention

1. Automated (i.e., data requirements and retrieval are designed to be automatically uploaded by the program 110 from the patient EMR (i.e., PACS 30) and individual Profiles database 113, 114 (i.e., device, patient, institutional and individual provider profiles));

2. Embedded (i.e., identification data specific to each individual device is directly embedded within each device, which can be retrieved by the program 110 from a universal “look up” database 113,114 for device specific data. In addition, the embedded identifying data can also correspond to an easily understood symbolic language (akin to pottery watermarks) for direct identification related to manufacturer, model, clinical application, and year).

3. Standardized (i.e., all data is confined to a standardized set of variables in order to create a referenceable database 113, 114 which allows statistical analysis across large numbers of devices, providers, and patients).

4. Intelligent (i.e., using computerized methods of artificial intelligence the program 110 can detect data outliers and automatically generate a query for clarification and data verification. If a data outlier is verified as correct by the program 110, this can also be used by the program 110 to automatically generate a QA analysis and alert in order to avoid a poor clinical outcome).

5. Interactive (i.e., based upon analysis by the program 110 of patterns of usage and an individual's profile, data query).

6. Customizable (i.e., analytics and automated alerts/prompts performed by the program 110 can be customized in accordance with institutional or individual needs).

7. Portable (i.e., readily accessible by data being held and retrieved by the program from the cloud).

8. Secure (i.e., database 113, 114 is routinely monitored and audited by the program 110; only authorized individuals can access data using the program 110, in accordance with their predefined privileges).

9. Involuntary (i.e., all parties and devices must actively participate).

10. Accountability (i.e., specific to context (procedure), device, clinical variables (patient), provider (institutional and individual levels)).

Medical Device, Provider, and Patient Registration

In one embodiment, two components of the present invention include an identification device embedded within each individual medical device, and the comprehensive database 113, 114 which is derived from standardized data attributable to the medical device, procedure being performed, service providers, and patient.

The identification device which is embedded and/or attached to the medical device includes a standardized method of identifying each individual medical device in accordance with a number of the following specific attributes.

A. Medical Device Identification Attributes

1. Type of device

2. Associated anatomic structure/organ system

3. Manufacturer (company name, location of production)

4. Model number and name

5. Quality control (identification of responsible party)

6. Temporal data (date/time of completion, shipping, expiration)

7. Clinical indications

8. Guidelines for Usage/Insertion

9. Purchasing data (seller and purchaser identification, date/time of purchase order, price)

10. Storage/inventory data (identification and physical location of organization receiving shipment, identification, acknowledgment, and date/time of person/s accepting receipt)

These individual data attributes can be stored in the database 113, 114, in one of two ways: 1) either directly within the identification device (i.e., internal storage 113), or 2) external to the device in an external database 114. In the event that an external database 114 is used for this data, the identification schema would serve as a device specific identifier, which could be directly correlated with device-specific data contained within the external database 114. In this case, the identification marker (e.g., RFID, engraved alpha numerics) would be unique for each individual device and when this unique ID is entered into the corresponding database 114, all associated data specific to that individual device would become accessible. In order to create a standardized medical device identification system (with associated device specific data attributes) an industry standard would be created by the program 110, analogous to the DICOM standard used in medical imaging, in which all industry participants would utilize the same identification schema and associated standard data elements. The concept of engraved identification markers would in some respects be analogous to that of watermarks used in pottery, in which a manufacturer uses a standardized set of engraved symbols to denote the company, location of origin, style, model, and year of production. In this case, a more elaborate set of identification markers could be created (which could even be microscopic in order to accommodate to physical size restrictions), which correlate to the attributes listed above, along with a unique identifier for each individual device.

In addition to the primary purpose of providing a unique identifier for each individual device, the identification system (and associated technology) of the present invention can also provide a number of other functional features aimed at security and counterfeiting. The device identification methodology can be simultaneously embedded within both the device and its external packaging. This provides a functional method of ensuring that the device has not been opened since the time it was packaged and shipped from the manufacturer to the end-user. By simply matching the identification markers on the external package and device, one can document and confirm authenticity and the lack of tampering. An anti-tampering mechanism could be incorporated which effectively disrupts the external packaging identification marker once the packaging has been opened, which effectively prevents opening and resealing of the medical device packaging prior to use. This security feature can be integrated into the corresponding medical device database 113, 114 by requiring the end-user to enter both the packaging and device identifiers into the database 113, 114 prior to use. In order to avoid either non-deliberate or deliberate human entry error, the entry of these identifiers can be automated through the use of an electronic scanner, which directly populates the packaging and device identifiers into the device database 113, 114. If these identifiers do not directly match with one another, or if this data is not successfully entered into the database 113, 114, then subsequent data related to the device and procedure being performed will not be recorded, which can prevent registration of the procedure and subsequent billing/reimbursement. (This ability to directly tie billing and reimbursement to successful medical device database 113, 114 completion, is another important function of the invention, and can be automated once all relevant device/procedure data has been completed and verified.). In more expensive and technology intensive medical devices (e.g., surgical hardware, cardiac pacemaker), the device identification system can also incorporate a locking mechanism (for security purposes), which effectively prevents access to the medical device until the identification process has been successfully completed. This would include an electronic locking mechanism which would require completion of the device identification process before it is unlocked and available for use. In addition, one could incorporate an additional requirement for successful patient and provider registration before the device would be unlocked. Due to physical and economic restrictions, this locking system would in all likelihood be restricted to larger and more expensive medical devices.

In one embodiment, the various data contained within the medical device identification schema can be automatically downloaded into a centralized Medical Device Database 113, 114, which in effect, creates a formalized registration process each time a device is used and a medical procedure performed. In addition to registration of the device, additional requirements for registration include the patient on whom the procedure is to be performed, and the various providers who will be participating in the procedure and usage of the device. These combined registrations of the device, patient, and providers serve a number of functions related to security, quality assurance, clinical outcomes analysis, creation of and compliance with best practice guidelines, and informed consent.

In one embodiment, patient registration includes both an identification/authentication process as well as downloading of pertinent clinical data which can be found within the patient's electronic medical record (EMR). The list of data elements included in the patient registration process are listed below, and collectively form the Patient Profile.

B. Patient Profile Data Elements

1. Demographics (age, gender, religion, ethnicity)

2. Education (education, language literacy, healthcare literacy)

3. Compliance (adherence to medical directives, communication skills, motivation)

4. Medical Problem List (active clinical diagnoses)

5. Pharmacology (current medications, drug allergies)

6. Interventional History (past surgeries, previous procedures, resulting complications, clinical outcomes)

7. Physical (size, weight, mobility, vascular access)

8. Lifestyle (diet, exercise, level of daily activities)

9. Genetics (family history, genetic predispositions)

10. Support (available support system, family/friend involvement)

The data contained within the collective Patient Profile can be used to create a standardized “Patient Profile Score”, which in turn can be used to quantify overall medical morbidity and procedure-specific risk. For the purpose of quantifying procedure-specific risk, individual data elements within the comprehensive Patient Profile can be selectively weighted in accordance with their contribution to procedural risk/benefit analysis.

This profile in essence is an encapsulated composite of the patient's medical record which is applicable to the medical procedure being performed. Demographic, physical, cognitive, and clinical attributes of the patient are contained within this profile which provides healthcare providers with an updated review of the patient's well-being and risk profile relating to the medical procedure to be performed. In addition to human review, computerized analyses and decision support can be performed by the program 110 to create a computerized risk/benefit analysis of the planned procedure by correlating medical device, patient profile, and provider profile data (which are individually and collectively contained within the Medical Device Database 113, 114). This program 110 derived Medical Device/Procedural Risk Benefit Analysis takes into account the historical performance (i.e., quality and safety) records of the medical device and provider specific to the planned procedure, along with the inherent clinical risk profile of the patient (in both general and procedure specific measures). A detailed discussion of the Medical Device/Procedural Risk Benefit Analysis is provided below.

In one embodiment, provider registration is another required prerequisite of the medical procedure and also entails an identification/authentication of each individual healthcare provider who is taking part in the planned procedure. Once the provider has been authenticated, their specific Provider Profile will automatically be retrieved by the program 110 from the master database 113, 114 and reviewed to ensure that they are qualified and credentialed to participate in the planned procedure.

C. Provider Data Elements

1. Education and Training (professional education, specialty training, licenses, continuing medical education)

2. Clinical Experience (years in practice, practice type, patient population served)

3. Affiliations (institutional affiliations, societal memberships, credentials)

4. Technical Skills (certifications, procedural experience, technical proficiency)

5. Outcomes Analysis (procedural clinical outcomes, adverse events, technical failures)

6. Communication (patient education, supervision of support staff, reporting and documentation)

7. Malpractice (iatrogenic complications, litigation, compliance with professional standards and guidelines, loss or restriction of privileges or licenses)

In addition to review of the provider's licenses and credentials by the program 110, other analyses derived from the Provider Profile database 113, 114 by the program 110 provide for historic analysis of the provider performance (i.e., quality and safety metrics) specific to the planned procedure and medical device to be used. In addition to this performance analysis, any relevant disciplinary and/or medico-legal actions are reviewed by the program 110 to assess procedural risk, and are incorporated by the program 110 into the Medical Device/Procedural Risk Benefit Analysis. The resulting data is incorporated by the program 110 into the Patient Informed Consent so as to provide the patient with reference quality and safety data specific to both the procedure and provider. In addition, if analysis by the program 110 of the Provider Profile database 113, 114 identifies a data outlier (e.g., provider not properly credentialed, has insufficient experience, unexpectedly high number of adverse events) then an automated alert is issued by the program 110 notifying the responsible parties of the cause for concern. If the data outlier is of sufficient concern to merit termination of the planned procedure by the program 110, then a formal reconciliation process is required before the procedure can go forward. This provider reconciliation process may take a number of forms including (but not limited to) replacement of the provider, addition of a supervisory physician, modification of the procedure, postponement of the procedure, or termination. This provider reconciliation process can only be circumvented in the case of a medical emergency where delays or postponement of the procedure risks patient death. Similar registration processes are required for all healthcare professionals taking part in the planned medical procedure (e.g., technologist, nurse, resident). The standardized method of electronic registration for the database 113, 114 could be performed in a variety of ways including biometrics, speech analysis, and unique data identifiers.

D. Medical Device/Procedural Risk Benefit Analysis

The data analytics derived by the program 110 from the database 113, 114 are described herein, and include a number of clinical outcome measures relating to safety and quality. Quantifying risk for a given procedure/medical device is highly variable and dependent upon a number of factors specific to the individual patient, provider, and procedure type. While a specific procedure (e.g., cardiac pacemaker insertion) may be relatively straightforward and low risk for one patient, it may have a much higher risk for another patient due to a variety of mitigating factors (e.g., comorbidities, body habitus, age, mobility). At the same time, attributes specific to the individual clinical provider may also play an important role in quantifying risk based upon technical proficiency, clinical experience, and education/training. The third component in risk analysis is the procedure being performed and the specific device being selected. Subtle differences in device design may have varying degrees of associated risk and this needs to be accounted for in the overall procedural risk.

While cumulative risk can be quantified in a number of different ways, one method is for the program 110 to create a generic procedure risk from longitudinal outcomes analysis of the database 113, 114. By correlating the frequency and severity of adverse events associated with individual procedures, a generic procedure-specific risk score can be created by the program 110. When this procedure risk score is in turn correlated with the individual device by the program 110, patient, and provider risk profile scores, one can essentially create a customized procedural risk.

Using the same methodology, the potential benefit of a specific procedure can also be quantified. The generic procedural benefit can be quantified by the program 110 by analyzing procedure specific outcomes data within the database 113, 114. This generic procedure benefit score can in turn be modified by the program 110 in accordance with individual device, patient, and provider procedural benefits. If these individual procedure-specific risk and benefit scores are created using a standardized Likert scale (on a scale of 1-5), they in turn could be combined by the program 110 to create a standardized Device/Procedural Risk Benefit Ratio. As an example, if a given Device Procedural Benefit score was determined by the program 110 to be 4 out of a possible 5 (i.e., high benefit) and the corresponding Device Procedural Risk Score was 3 out of a possible 5 (i.e., intermediate risk); then the combined Risk/Benefit Ratio would be 3/4 or 0.75. Using this scoring methodology any ratio less than 1 would be considered to have a beneficial risk/benefit analysis, whereas a ratio greater than 1 would have a poorer risk/benefit analysis.

Another advantage of using outcomes data from the database 113, 114 is the ability to modify the selection of different procedures and devices in an attempt to improve the Risk/Benefit Analysis. As an example, suppose a physician is contemplating performing a lung biopsy on a patient for diagnosis of a pulmonary nodule. Based upon the analysis of the proposed procedure and biopsy device, a poor risk/benefit analysis score is determined by the program 110. In an attempt to improve this risk/benefit analysis, the physician selects two other biopsy devices to see if there would be a theoretical improvement in the risk/benefit analysis. In doing so, the provider identifies a device with an improved score and in response, chooses to select that specific device for the planned procedure. Alternatively, the physician could see if modification of the procedure could yield an improved risk/benefit score (e.g., changing the procedure from a biopsy under fluoroscopic guidance to a biopsy under CT guidance). While the CT guided procedure in general, has an improved risk/benefit profile than the fluoroscopic procedure, this procedural improvement is not realized when reviewing data specific to the individual physician's profile (i.e., who uniquely demonstrates improved risk/benefit using fluoroscopy). As a result, the physician chooses to stick with the procedure as planned, but switch to an alternative biopsy device with a higher risk/benefit profile.

E. Dynamic Assessment of In-Vivo Device Positioning, Integrity, and Functionality

One important feature of the present invention is the ability to assess real-time medical device performance in vivo. While medical device performance can be defined in a number of ways, the three major variables which will be used for measurement and analysis are 1) device positioning, 2) integrity, and 3) functionality. Device position refers to the specific anatomic location in which the medical device is located within the human body, and the relationship between this “actual” position to that of an “optimal” position. This “optimal” position is defined as the ideal location for device position, in order to optimize device/patient safety, functionality, and clinical outcomes. An additional variable to analyze when assessing device position is the “margin of positional error”, which is specific to each individual device and represents the maximum distance (in three-dimensional space) between the “actual” and “optimal” device positions, which will allow for an acceptable level of device functionality and safety. This margin of positional error can be customized to the specific clinical indication and patient, in accordance with the indicated use and specific patient anatomy.

To illustrate how these device position measurements and analytics are used, examples of an intravenous catheter and aneurysm coil are used. For the intravenous catheter, the determination of optimal position and margin of positional error are specific to the anatomic location of the catheter, patient attributes, and its intended clinical use. If the intravenous catheter was being inserted for the routine administration of intravenous medication via the right internal jugular vein in a normal size, relatively healthy adult male patient than the optimal position would be determined to be the distal superior vena cava with a margin of positional error of 5 cm. If, however, we were to change the patient attributes to that of a pediatric (6 year old) male using the same catheter, entry site, and clinical usage, the margin of positional error would now be 2 cm (reflecting the relative size differences between these two patients). Alternatively, one may modify the intended clinical use in the original adult male patient from that of routine administration of intravenous medication to that of specific administration of chemotherapy medications, which require a lower margin of positional error to only 2 cm (as opposed to 5 cm for more general all-purpose use). This reflects the ability of device positional measures and analytics to take into account the specific medical device, patient attributes, relevant anatomy, and clinical use.

In another example, such as an intracerebral aneurysm coil, one would arrive at a far different margin of positional error, due to the type of device, relevant anatomy, and clinical use. Unlike an intravenous catheter which will often demonstrate variability in position within, affecting clinical use or patient safety, an aneurysm coil must have a fixed location, devoid of positional variability in order to function properly. At the same time, if this aneurysm coil was located within a brain aneurysm, even the slightest change in position could be life threatening, due to risk of intracerebral hemorrhage and subsequent death. As a result, the margin of positional error for this intracerebral aneurysm coli may be determined to be 1 mm. In the event that a larger positional change of 2 mm was recorded into the database 113, 114, an automated notification pathway would be instituted by the program 110 to alert the provider of a potentially adverse clinical outcome and need for further evaluation (e.g., brain CT or MRI).

Over time, these sequential measurements of device position will provide one with the ability to track device positional changes over time specific to the individual device, patient, and clinical use. If the aforementioned intracerebral aneurysm coil was found over a 2 year period of time to demonstrate positional changes of 0-2.5 mm without an adverse clinical event, then the device profile may be modified to reflect a modified margin of positional error from the original 1 mm to a new 2.5 mm. This illustrates the ability to dynamically adjust medical device profile practice guidelines in accordance with general community-based medical standards, along with the specific data measurements and analytics of the individual patient. In this example, by modifying the margin of positional error to 2.5 mm, the accompanying automated notification pathway would be similarly adjusted so that positional measurement changes of <2.5 mm would no longer result in an automated provider alert by the program 110. Note that these automated device positional alerts can also have device-specific embedded decision support tools. In the example of the aneurysm coil with a measured positional change exceeding the defined margin of positional error, an automated order for brain CT could be generated by the program 110 upon recording of the excessive measurement. This serves to standardize and streamline medical care, while also providing for consistent outcome data which can be incorporated into the medical device database 113, 114 for outcomes analysis.

The second major variable used for medical device analysis is device integrity, which represents a standardized method for quantifying the components of an individual device remain intact and functional. Analysis of device integrity includes assessment of the location of integrity loss (i.e., the specific location and/or medical device components involved), the severity of the integrity loss (i.e., the magnitude of the malfunction), and the clinical ramifications (i.e., the degree to which loss of device integrity will adversely affect clinical care and outcomes). Just as was the case for assessment of device positioning, each individual medical device will have its own unique device profile, which will be in part related to clinical use and individual patient attributes.

To illustrate how measurement and analysis of device integrity is used, one can take the example of an inferior vena cava (IVC) filter which is inserted for the treatment of lower extremity deep venous thrombosis (i.e., blood clots). In this example, one of the IVC struts which attaches to the IVC walls has been detected to have a loss of integrity, such that a single strut has been broken and is separated by a 1 mm gap with the central component of the IVC filter (note: this loss of integrity can be established by sensors embedded in the IVC components). In this specific type of IVC filter, there are a total number of 8 struts (4 on each side), whose primary purpose is to ensure that the IVC filter remains embedded within the walls of the IVC and is not dislodged or significantly altered in position (which could potentially prevent it from trapping embolic debris in the bloodstream and resulting in a left threatening pulmonary embolus). With all other 7 struts determined to be intact and with an integrity gap of only 1 mm, it is determined that the IVC filter remains functional and is not in need of repair and/or replacement. Note that the determination of integrity loss severity can be established in a number of ways including (but not limited to) community established standards, mechanical testing of devices, meta-analysis of the medical device database 113, 114, and longitudinal device and patient-specific measurements. This latter component is an extremely valuable method of analysis for it utilizes patient and device specific integrity data over time, which can be correlated by the program 110 with other device specific data (e.g., positional change). Using this same example, suppose the IVC filter with a single broken strut and a 1 mm gap is tracked over time and found to remain stable (i.e., no additional integrity loss). While the integrity has remained unchanged, however, there has been a noticeable change in device positioning over time with a change in device positioning of 3 mm on the side of the broken strut. When correlating with 3-D computerized simulation software mapped to CT angiography, this 3 mm of positional change in the device produces a 1 mm gap in the filter/IVC lumen interface, which was not previously demonstrated. This in effect means that small (<1 mm emboli) can pass through the IVC at the point of the filter/IVC interface and travel to the lungs (i.e., pulmonary emboli). This example illustrates how the device integrity data can be correlated by the program 110 with additional data contained within the database 113, 114 and used for customized clinical and safety analysis.

For example, suppose that the patient with the IVC filter has experienced similar problems with IVC filter integrity in the past. Two previous IVC filter failures have been recorded, each of which has been associated with damaged struts requiring replacement. The combination of multiple IVC filter failures and similarities in integrity failure would suggest that some intrinsic problem with the patient's anatomy or medical device design are contributing to poor clinical outcomes. The ability to mine data within the database 113, 114 provides a methodology for comparative assessment of medical devices specific to the patient profile, clinical use, and specific device integrity characteristics. One could effectively search medical device integrity data for the purpose of identifying IVC filters with the highest integrity analytics, and specifically evaluate integrity deficiencies related to strut integrity. In addition to seeking out IVC filters with high integrity measures, one could further analyze specific patient characteristics (e.g., morbid obesity) and the correlation with IVC filter integrity. In this example, three different types of IVC filters were found to have higher integrity measures than their peers (with particularly low strut failure rates). When cross referencing these 3 IVC filter integrity measures with patient profile characteristics specific to the patient of record (e.g., morbid obesity, IVC diameter of 4 cm), one of the IVC filters was determined by the program 110 to have the best overall analytics, and was therefore, chosen as the medical device of choice. This illustrates the ability of the program 110 of the present invention to assist in comparative analysis of medical devices specific to the device performance analytics and individual needs and attributes of the patient.

The third major variable is device functionality. Even if a medical device is found to be properly positioned and intact, its utility is limited if it is not fully functional. A detailed discussion follows relating to how functionality of a given medical device can be measured and recorded in the database 113, 114 for longitudinal analysis and intervention. One important feature of the present invention is for the program 110 to create a standardized set of metrics which can be used for functional analysis of each individual medical device, and used to assist in device selection based.

As previously stated, device positioning can be longitudinally monitored and analyzed by the program 110 using a series of sensors embedded in the inner and outer surfaces of the device throughout its footprint. This provides providers with an accurate and real-time assessment of device position changes over time, which can be correlated by the program 110 with physiologic and mechanical changes. From a physiologic perspective, minute to minute changes in human physiology change, which can alter a given device's position and functionality. Examples may include (but not limited to) a cardiac valve (which is routinely subjected to changes in blood flow and heart mechanics), an intravascular stent (which is subject to blood pressure changes), and a spinal implant (which is subject to changes in mechanical stress with physical movement). These “routine” or baseline positional/functional changes in medical devices can be analyzed by the program 110 using real-time derived sensor data, recorded in the medical device database 113, 114, correlated with relevant physiologic data (e.g., blood pressure, heart rate, left ventricular wall motion and end diastolic/end systolic pressure measurements), and longitudinally analyzed (i.e., device positional change over time). The analysis and feedback of this device positional and functionality data can be automatically transferred by the program 110 at predefined time intervals (e.g., weekly) to authorized providers, along with the ability to automate data alerts and prompts when predefined data thresholds are exceeded, thereby requiring receipt notification, acknowledgement, follow-up, and potential intervention.

In one embodiment, the baseline device position is first established at the time the device is implanted/deployed within the body. At this time, sensor activation and function is verified through active data recording and testing (i.e., sensor quality control). The recorded data can in turn be used by the program 110 to create an in-vivo three-dimensional anatomic map of the device and its surrounding milieu. By continuously recording this sensor positional over a defined period of time, a real-time 3-D device map can be created which visualizes the device in vivo, while also quantifying device positional changes over time, along with corresponding physiologic and/or mechanical changes. Note that these baseline mechanical changes can be established through both “passive” and “active” methods. In passive operation, baseline device positional change is recorded without any provocative patient action and/or movement. In “active” assessment of device positional change, a patient may be subjected to a defined set of actions which are designed to place some degree of mechanical stress on the device and its surrounding anatomy (e.g., active flexion and extension, twisting, and/or rotation of the spine in the setting of an indwelling surgical device or implant (e.g., spinal fusion, pedicle screws, surgical rods). The net result is the 3-D sensor derived “device map” can be recorded by the program 110 at baseline and subsequently reanalyzed by the program 110 over time to assess changes in device position and/or functionality.

In some situations, one may elect to perform a medical imaging examination (e.g., CT, MRI) to correlate the sensor derived device map with that of an external data source. These 2 and 3-D volumetric medical imaging exams can provide an excellent and easily performed reference to the sensor derived data and assist in analysis of device position and functionality over time. As an example, immediately following the interventional procedure (e.g., placement of an endoluminal stent graft (see FIG. 9) in the abdominal aorta for the treatment of an abdominal aortic aneurysm), CT angiography can be performed (with and without intravenous contrast administration) to assess stent positioning and functionality (i.e., stent patency, stent leakage). This data can in turn be correlated by the program 110 with the baseline sensor derived map to ensure that the two separate data sets are synchronous to one another. In the event that there is any discrepancy in these datasets (e.g., failure of sensor derived data in a specific location of the device), a baseline data adjustment can be made to accommodate this discrepancy, thereby allowing future data and analyses to be consistent with any baseline data discrepancies. At the same time, if sensor malfunction was recorded over time (e.g., new sensor failure), having the ability of the program 110 to correlate real-time sensor and 3-D imaging data would provide a valuable means of data accommodation.

Since the sensor derived data may be more detailed in its analysis by the program 110, when compared with an imaging exam (i.e., microscopic versus macroscopic positional analysis), it would not be unexpected for subtle device positional change to become apparent at an earlier point in time than correlating imaging data. If for example, a single strut of an inferior vena cava (IVC) filter became dislodged from the IVC wall, it would be expected to be detected by the program 110 in the sensor data but not necessarily visualized on the CT imaging data. This is in part due to the fact that the data derived from the sensors is far more granular as well as the fact that sensors can be distributed (and anatomically localized) throughout the entire surface of the device. The distribution of sensors on the device can in effect create a “3-D sensor distribution map” which is extremely valuable in data analysis, relating to the specific location of the device and its associated positional/functional data. In the example used of a broken strut in an IVC filter, one can literally identify the location, severity, and type of malfunction based upon comparative sensor data analysis. To illustrate how this would work, one example is an IVC filter which is situated in the IVC at the L3 level. On CT imaging, the IVC filter remains in stable positioning and without interval change when compared with the baseline post-procedure CT exam. The sensor derived data tells a different story, because of the inferior right sided struts has been broken but not detached from the core filter device. In essence this subtle non-displaced break is non-perceptible on routine CT imaging. The sensor derived data would by analyzed by the program 100 which would provide an alert to the provider to the fact that there is a newly detected incongruity in the device (i.e., 1 mm separation between two adjacent sensors), but no change in positioning of the peripheral sensors attached to the distal struts which are located along the walls of the IVC. Therefore, the overall position if the IVC filter (as detected by its position relative to the IVC walls) remains unchanged, and therefore appears unchanged on serial CT exams. If the device had a total of 200 sensors embed within its architecture and only 1 sensor was recorded to exhibit a positional change, then the data would suggest the device malfunction was minor at this point in time. However, due to the specific location of this involved sensor (i.e., at the attachment between the strut and filter core), the potential for increased positional change and or device malfunction would be fairly high. This would therefore, call for increased scrutiny of sensor data collection and analysis, so that any future worsening and/or involvement of additional adjacent sensors may serve as a prompt for intervention (e.g., filter removal and replacement). If over time, the initial separation of 1 mm was to increase to 2 mm and two adjacent sensors became involved, the device malfunction would be elevated to a higher clinical status and degree of scrutiny by the program 110. The corresponding 3-D sensor distribution maps generated by the program 110, would show the location of the involved sensors along with the specific type and severity of the defect. When correlated by the program 110 with the 3-D medical imaging datasets, one could begin to improve visualization of device malfunction on CT and use the sensor maps to create advance image processing algorithms and software to improve medical device visualization.

If one were to correlate physiologic and sensor data, one could begin to have an objective methodology for more effectively analyzing functionality and interventions strategies. In the previous example, the small defect in the IVC filter (i.e., 1 mm break at the strut/core interface) would not be expected to be significantly affected by physiologic change, since venous pressure measurements are relatively low, thereby subjecting the filter to minimum external pressure variation. If, however, one was evaluating a stent in the arterial system instead, the increased arterial pressure measurements may create higher mechanical stressors on the device, which may in turn accelerate device breakage and its clinical severity. The same 1 mm gap between adjacent sensors in an arterial stent may therefore cause greater clinical concern and need for intervention, which would be dependent upon the type of device and its specific arterial location. Now if we were to go one step further, suppose the arterial stent is located in the abdominal aorta and the patient in question has severe hypertension (e.g., 260/120 mm Hg). The markedly elevated blood pressure (normal is below 140/90) would cause increased mechanical pressure on the arterial stent and create greater concern for a given tent deficiency when compared with a patient who has normal blood pressure (e.g., 120/80 mm Hg). In addition, this specific patient may be prone to fairly dramatic changes in blood pressure so that intermittent fluctuations may occur which may include a high of 300/150 mm Hg. By having the ability to correlate real-time sensor and physiologic data, the program 110 can effectively create an analysis of device malfunction over time in accordance with blood pressure variation. Suppose in this example, the sensor detects a defect of 1 mm with a blood pressure measurement of 180/100, which increases to 1.4 mm at 260/120, and 1.7 mm at 300/150 mm Hg. Over the course of 4 weeks, however, these same measurements increase to 1.3 mm at 180/100, 1.8 mm at 260/120, and 2.4 mm at 300/150 mm Hg. This longitudinal data shows that the device defect has slightly increased at lower blood pressure measurements over time and the defects have progressively worsened at higher blood pressure measurements. This ability of the program 110 to correlate device positional change and function over time with physiologic measures provides an important method of analysis and establishment of best practice guidelines specific to the individual patient. By the program 110 having the ability to utilize this data in the database 113, 114 and cross reference with large numbers of different patient profiles, one can effectively create an objective method of using device positional and functional data to guide therapy and interventional strategies.

Another important use of this sensor data is the ability to analyze defects specific to individual types of medical devices. In contemporary practice, only major device defects are routinely detected and when these require device removal and/or replacement, this information will not routinely be recorded into a master database 113, 114. Using the invention, all device specific data is recorded by the program 110 in the master medical device database 113, 114, which in turn can be used for comprehensive analysis of device usage, functionality, safety, and patient profiles. In addition to documentation of major device malfunctions, the aforementioned sensor derived data can provide an objective means for early and subtle detection of device malfunctions, which can be used to improve clinical outcomes, decision support (in device selection), and technology refinement.

This ability of the program 110 to objectively record, quantify, and analyze device integrity, positioning, and functionality in real-time can also be enhanced by the ability of the program 110 to perform comparative analysis with “comparable” data contained within the database 113, 114. “Comparable” data can correspond to the specific device attributes (e.g., manufacturer, model, device category), patient profile (e.g., age, size, comorbidities), clinical use (e.g., indication for use, underlying disease, severity of illness), individual provider profile (e.g., clinical experience, education/training, technical skills), and institutional provider profile (e.g., type of institution, patient population served, support staff, technology in use). This ability of the program 110 to correlate real-time device data with “comparable” data provides a method for predicting the clinical and technical ramifications of a given device measure, as determined by historical device data use within comparable peer groups. If, for example, a defect in device integrity is recorded in an orthopedic prosthesis (e.g., hip arthroplasty), it is critical that the patient and clinical provider be aware of the deficiency, and the program 110 can determine risk factors for device failure (e.g., excessive stressors in daily use), and objectively analyze the inherent risk associated with passive (e.g., continued device surveillance) versus active interventions (e.g., arthroplasty removal and revision). In conventional practice, this risk assessment is customarily performed largely based upon the individual provider's experience (which represents a relatively small sample size of patients and devices) and data within the medical literature (which is primarily comprised of small generalizable data pools which do not take into account individual attributes specific to the patient, clinical use, provider, or individual device). In addition, (and perhaps most importantly), this collective provider experience and medical data decision making is largely predicated upon overt device deficiencies, not the subtle earl deficiencies which are recorded by the program 110. As a result, the present invention and its program-derived data analytics provides a method for proactive, preventive medical care, as opposed to reactive, corrective action. This proactive approach is only feasible when objective and standardized data can be recorded and analyzed by the program 110 throughout the lifetime of the medical device. By having the ability of the program 110 to correlate this real-time data with “comparable” longitudinal data, best practice guidelines can be created which are customized to the specific device, patient, provider, and clinical scenario.

An example of how this customization of medical device data can be applied is in the setting of 3 different patients each with the same specific hip prosthesis and the same recorded deficiency (e.g., 2 mm of loosening at the distal tip of the prosthesis). In the first example, the patient is a 35 year old laborer who performs a great deal of strenuous activity at work. In the second example, the patient is a 62 year old retired female who leads a relatively sedentary lifestyle and whose main form of exercise is daily walking of 2-3 miles. The third example is an 84 year old male with severe emphysema and heart disease, whose daily activities are largely restricted to his home. Given the same medical device and deficiency, one may assume that the treatment plan would be relatively similar. However, there are two fundamental advantages of the present invention which provide an improved ability to customize treatment and improve clinical outcomes. The first of these advantages is the ability of the program 110 to collect real-time device data which can be directly correlated with activity and device related stress. As each patient undergoes their routine daily activities, the program 110 will record the sensor derived data in the devices which track the device positional changes over time, and which can be correlated with time stamped activity data (e.g., external sensors embedded within clothing, patient recorded daily logs). The second advantage offered by the invention is the ability of the program 110 to correlate this real-time device data with comparable data in the database 113, 114 to determine how alternative treatment strategies fared given different patient, clinical, and provider profiles for the collective population of patients and devices.

Using this example of three different patients (with the same type of medical device and device deficiency), one can see how the data can be used by the program 110 to customize treatment in accordance with best practice and clinical outcomes data. In the first patient (35 year old highly active male), the real-time data demonstrated relatively high degrees of positional change in the prosthesis with active hip flexion. When correlated by the program 110 with longitudinal data from the database 113, 114, this increased positional change was associated with high degrees of prosthesis failure, despite the relatively small degree of baseline positional change. Given the patient's desire to continue their current level of physical activity, it was determined that the best course of action was to replace the existing arthroplasty with one that has a high degree of positional stability, specifically in the current problematic area (i.e., distal femur). A search of the database 113, 114 by the program 110, identifies three specific prosthesis candidates which demonstrate high performance measures for this specific concern. Further analysis is performed by the program 110 to compare prosthesis life spans (i.e., time before prosthesis replacement is required) and durability for high levels of activity. Based upon this comparative prosthesis analysis, one prosthesis was identified by the program 110 to be the best candidate based upon its longer life span and high levels of durability for high intensity and prolonged physical activity. The one downside associated with this ideal prosthesis candidate was the significantly higher cost of this prosthesis in comparison to the two other prosthesis candidates. Having the ability of the program 110 to utilize real-time patient and device specific data along with comparative analysis of the database 113, 114, provides objective and compelling data to provide authorization from the patient's payer (i.e., health insurance provider). This illustrates another compelling application of the invention, in its ability to provide clinical, technical, and economic analysis of medical devices in order to perform objective cost-benefit analysis and optimize healthcare outcomes in a cost efficient manner.

In the second patient (62 year old retired female with moderate levels of low impact exercise), the same medical device and deficiency may be associated with a different treatment plan. Using the same strategy of analyzing device positional change over time and correlating with physical activity, minor degrees of prosthesis positional change are documented by the program 110, which are symptomatic when correlating with the patient's daily logs. While the relatively minor positional change measurements would likely warrant conservative management, the fact that these small positional changes correlate with patient reported symptoms (which is another unique attribute and application of the invention—namely, the ability of the program 110 to correlate real-time objective device data with synchronous patient subjective data), may affect treatment strategy. In the absence of symptoms, treatment would likely include continued device surveillance, in order to ensure that no interval increase in device positional change occurs. As long as the device positional change remains relatively constant and small, no additional intervention is planned. However, in this case, the presence of patient reported symptoms which directly correlate with increased (albeit minor) device positional change would place this patient into an elevated intervention category. When correlating the device and patient data with “comparable” data from the database 113, 114, the program 110 analyzed that “comparable” patient treatment with physical therapy (e.g., hip mobility and strengthening exercises) were found to have a reduction in symptoms and reduced requirement for prosthesis revision. As a result, the patient was conservatively managed with a combination of physical therapy, heightened data surveillance, and activity modification in an attempt to prolong the lifetime of the prosthesis, reduce symptoms, and maintain prosthesis functionality (i.e., positional change over time).

The third patient example includes an 84 year old male with severe emphysema and heart disease, whose daily activities are largely restricted to his home. Since this patient's level of activity is negligible, there is essentially no device positional change throughout the course of a given day. Over several months, however, the degree of device positional change increases from its baseline of 2 mm to 5 mm, which would typically indicate device instability and requirement for prosthesis revision. The orthopedic surgeon provider is, however, concerned about the patient's ability to endure such a surgical procedure given his comorbidities and limited life expectancy. By the program 110 analyzing comparative data in the database 113, 114, statistical analysis can be performed which estimates the risks associated with surgical revision versus conservative management. Through such an analysis by the program 110, it is determined that the risk associated with surgery and post-operative physical therapy exceeds the risk of maintaining the existing prosthesis. In order to reduce the risk of the existing prosthesis (e.g., infection, fracture), an occupational therapist is consulted to assist teaching the patient the most effective method of ambulation and transfers, within his everyday living environment. Ironically, the selection of this occupational therapist consultation can be enhanced through analysis of the database 113, 114 by the program 110, which analyzes provider performance specific to the individual medical device, patient, profile, and clinical condition.

Another important application of the invention was briefly touched upon in a prior example. Along with sensor-derived device data, patient subjective data can in some circumstances play an essential role in medical device analysis. Medical devices which are intrinsically related to supporting anatomy (e.g., spine, extremities) and physiology (e.g., heart rate, respirations) may often produce noticeable symptoms to the patient, which in turn can be used to generate patient data input. In the previous example, the symptom of localized hip pain in the setting of hip prosthesis was used. Another comparable example would be that of pain or neurologic symptoms (e.g., weakness, sciatica, numbness, paresthesias) related to spinal hardware. Examples of device malfunction producing physiologic changes and symptoms may include a malfunctioning cardiac pacemaker producing chest pain or tachycardia (i.e., rapid heart rate), or malfunctioning tracheostomy tube producing tachypnea (i.e., rapid breathing) or shortness of breath. The ability to accurately record and track these symptoms are dependent upon the ability to record accurate and reliable time stamped data along with the specific symptom, location, and activity at the time of the event. While patient logs may be one method of accomplishing this, it is handicapped by the degree of patient compliance. It may not be feasible or practical to expect a patient will reliably, consistently and accurately record the requisite data. Alternatively, one could utilize technology to accomplish this task through either automated or manual data input. In the automated mode of operation, a wearable technology (e.g., smart watch, pulse oximeter) may continuously monitor data measurements (e.g., pulse, respiratory rate) and record time stamped data whenever the established baseline data (or defined threshold) is exceeded. At the time of this event, an electronic alert or trigger may be sent to the patient by the program 110, which in turn requires their additional input as to any specific symptoms they may be experiencing and the nature of the precipitating event (e.g., change in activity). This patient directed data input could be recorded by the program 110 in a variety of ways including (but not limited to) speech, text, or symbolic input. For speech, the patient could activate the speech input device integrated into a wearable technology (e.g., smart watch, necklace) and record the input data via an audio file, which can be subsequently transcribed using speech recognition software and transmitted by the program 110 to the database 113, 114. For text data input, the patient could type in corresponding data into their smart phone, which would in turn automatically transmit the time stamped data through wireless transmission to the database 113, 114. For symbolic data input, a standardized language using icons and symbols can be created, which provides the patient with the ability to select the symbolic language which corresponds to the symptoms experienced. Just as was the case with text input, this symbolic data input would be time stamped, recorded and transmitted to the database 113, 114 by the program 110. The geographic location of the patient at the time of data input could also be recorded using GPS technology. While this subjective patient input data is not critical to the functionality of the invention, it does provide an important ancillary source of data, separate from the sensor derived device data, which can play an important role in assessing the clinical significance of device related data, which may be highly variable for each individual patient. The ability of the program 110 to directly correlate time stamped and activity related device and patient data may prove extremely beneficial in defining best practice guidelines as relating to the specific medical device, clinical situation, and individual patient.

Another important feature of the invention is the ability to create “Smart” technology functionality using the real-time device and database 113, 114 data. The same concept of automated prompts and alerts to providers when predefined data thresholds are met, can also be applied to the patient. This serves as a means of alerting the patient to a potential problem related to the device as well as serving to elicit a response from the patient. In the previously described application where patient input data is required for the program 110 to correlate with device data at a certain point in time, is an example of such multi-directional communication. In the prior example, the sensor derived device data (i.e., hip prosthesis) recorded an increase in device positional change with active hip flexion. Since it is important to track the specific activity taking place at the point in time in which the device positional change is taking place, an automated alert could be transmitted by the program 110 to the patient (as well as the provider) to alert them of a potential problem, solicit input data regarding activity and symptomatology, and provide feedback for medical assistance. An example of this latter application (i.e., medical assistance feedback) could be when a cardiac pacemaker is malfunctioning and the physiologic data recorded by the program 110 indicates an abnormal heart rate and/or rhythm. The program 110 could send an alert simultaneously to both the provider and patient, which would include instructions related to medical care (e.g., proceed to the nearest hospital emergency room, initiate consultation with your cardiologist, cease any strenuous activity pending evaluation of the pacemaker). In addition, this “Smart technology” feature of the present invention could also prompt the program 110 to require a direct consultation between the patient and clinical provider.

One way (but certainly not the only way) to initiate a patient-physician consultation would be as follows:

1. Data derived from the device (e.g., cardiac pacemaker) is analyzed by the program 110, which detects an abnormality in device performance which exceeds a predefined threshold.

2. A repeat data analysis by the program 110 is triggered to confirm the data abnormality.

3. If the data abnormality is confirmed by the program 110, an automated analysis of the database 113, 114 is performed by the program 1110 to analyze the individual patient's historical data analytics referable to the specific device.

4. A simultaneous analysis of the database 113, 114 by the program 110 is performed to analyze “comparable” data (i.e., similar device, similar data abnormality, similar patient profile).

5. Based upon these “same patient” and “comparable patient” analyses, artificial intelligence and data mining techniques are used by the program 110 to predict clinical outcomes and interventions strategies.

6. The collective data analyses are then used by the program 110 to prioritize the degree of clinical severity related to the device malfunction (e.g., using a Likert scale of 1-5, where 1 is routine and 5 is life threatening).

7. Based upon the determined clinical severity grade, an automated alert is transmitted to the patient by the program 110, via electronic means (i.e., fax, email, text etc.), and to the provider via electronic means, notifying each party of the detected device abnormality, the assigned clinical severity, and follow up recommendations. (These follow up actions are based upon longitudinal outcomes analysis of the database 113, 114 by the program 110, along with customized input and modification from the provider and patient.)

8. The alert is transmitted to each party by the program 110 using a predefined communication pathway (i.e., electronic means). This customized communication pathway takes into account each parties' preferred method of communication (e.g., telephone, smart watch, smart phone) along with the clinical severity of the problem (which establishes the intervention requirements and time urgency).

9. Confirmation receipt of the alert is required by all involved parties which ensures that the message was received in a timely fashion, understood as to its content, and plan for follow action.

10. An option for patient-provider consultation is included in the notification pathway by the program 110 (and is mandated for higher levels of clinical severity).

11. When activated, the program 110 automated consultation application electronically connects both parties upon receipt confirmation, using the preferred method of communication (e.g., text, phone).

12. When an emergency status has been assigned to the device malfunction and recorded by the program 110, a GPS tracking system is activated by the program 110 within the patient smart device to provide guidance as to the patient's physical location over time. This will provide a method for emergency response professionals to track and seek out the patient, as clinically indicated.

13. The communication pathway can be deactivated by the program 110 when the device data returns to baseline or the patient has been directly assessed by an authorized clinical care provider (e.g., emergency room physician, primary care provider).

14. All relevant data is recorded in the database 113, 114 by the program 110 for future analysis.

15. In the event that initial communication was unsuccessful, an automated escalation pathway is activated by the program 110 to ensure timely receipt and follow-up action is taken, commensurate with the clinical severity of the device malfunction.

While the standard “smart” functionality of the present invention includes an automated alert or prompt based upon the identification of abnormal device data by the program 110, communication of a device related abnormality can also be initiated by the patient. Returning to the previously cited example of the pacemaker, the patient may experience a symptom they believe may be referable to a malfunctioning device (e.g., chest pain, irregular heart rhythm). The functionality of the program 110 of the present invention provides for a quick and easy method for patient data input to initiate a device data check and automated prompt of the provider. In this scenario, the patient inputs the symptom experienced (i.e., using speech, text, or symbol based data input) and a request for device evaluation. The resulting device related data is recorded and analyzed by the program 110 with respect to the database 113, 114. The resulting analysis of the program 110 is automatically transmitted to the patient, with a consultation option to the provider. In the event that the program 110 finds that the data is found to exceed the predefined threshold, the automated communication pathway is activated.

Another unique feature of the present invention is the (optionable) ability to share device related data with the device manufacturer. This becomes extremely important in the event that a technical problem of the device is recorded and the patient and provider are in search of additional knowledge (e.g., frequency of device malfunction, potential remedies, device replacement warranty). While the program 110 will collect all device related data and share anonymized data with authorized parties (e.g., regulatory agencies, manufacturers, researchers) to ensure patient safety standards are maintained, it is valuable for the program 110 to have the ability for direct data communication with the vendor to troubleshoot and offer guidance when device malfunction occurs. In effect, this would lead to the creation of “device hotlines” with vendors which provides patients and providers with the option to directly communicate and share device related data with the device manufacturer. Since all data communications and database 113, 114 analyses are recorded by the program 110, this would also serve as an early detection method for identifying device malfunction on a broad level, or assist in defining specific patient profile groups at risk for device malfunction. Device manufacturers' response to these communications will also be incorporated into the device profile and analysis by the program 110, thereby serving as an inducement for device manufacturers to be proactively involved in addressing device malfunction and technology refinement.

The responsiveness of medical device manufacturers to device related complaints, concerns, and questions could represent an important component of medical device analysis from the viewpoints of patients, clinical providers, third party payers, and regulatory agencies. Using the database 113, 114 and the application for reporting potential device malfunctioning, the program 110 would provide consumers with the ability to review the frequency and severity of device malfunction, the clinical and economic ramifications of these device deficiencies, and the timing and responsiveness of consumer related complaints. In essence, this could become an effective method of objectively analyzing both device and manufacture performance. Healthcare consumers (i.e., patents, providers, and payers) would gain by having direct access to comparative medical device data while device manufacturers would gain by having the ability to identify technical deficiencies at an earlier point in time along with the ability to proactively intervene (which could in the long term reduce insurance and legal costs related to malpractice).

Embedded Biosensors and Derived Analytics

In one example, a biosensor embedded medical device (e.g., esophageal stent, arterial stent graft, biliary duct catheter) is provided, which demonstrates the various real-time physical measurements which can be recorded and analyzed by the program 110 in vivo, to continuously assess device performance and functionality.

In one embodiment, the biosensor 200 (i.e., vascular catheter) includes an outer wall sensor 201, and an inner wall sensor 202.

In this example, the tubular shaped configuration of the medical device provides for well-defined entry and exit points. Using the example of an abdominal aortic arterial stent graft, the device is intended to bypass a segment of pathology (e.g., abdominal aortic aneurysm), while maintaining normal arterial flow throughout its course. A number of potential clinical and technical complications could result from device malfunctioning, which can be automatically detected by the program 110 at the onset, through continuous sensor derived data.

Sensors 201, 202 embedded throughout the internal and external components of the medical device can, in effect, constantly measure a variety of data elements related to both the physical characteristics of the device, its intrinsic functionality, and characteristics of the surrounding internal milieu. In the example of an arterial stent graft, this surrounding milieu would include the intrinsic physical anatomy (e.g., native abdominal aorta) and its internal anatomy (e.g., bloodstream contents). As a result, sensor derived data could detect a physical defect in the device (e.g., hole in the catheter), abnormality in the external anatomy (e.g., expansion of the abdominal aortic aneurysm), or deficiency in internal anatomy (e.g., blockage in blood flow). By having the ability of the program 110 to continuously track sensor derived data in real time, these abnormalities could be detected at a much earlier point in time than would be possible in conventional practice, by having the ability of the program 110 to identify early data trends which differ from established baseline measurements. In addition, the ability of the program 110 to perform sensor quality control (i.e., determine the intrinsic functionality of each individual sensor) in vivo, assists in differentiating between sensor failure and true pathology as the source of this data variation.

To illustrate the clinical functionality and diversity of medical device sensor derived data, the proximal end of the device, with arterial blood flow entering the stent graft, effectively demarcates the native abdominal aorta from the modified portion of the abdominal aorta containing the stent graft device. At the opposite (i.e., distal) end of the device, arterial blood flow exits the stent graft and re-enters the native abdominal aorta. In between these proximal and distal ends of the device is the body of the stent graft, which in effect, serves as a conduit for blood flow across the area of pathology (e.g., abdominal aortic aneurysm). Sensors embedded throughout the entire length of the device (both internal and external in location) can record a wide array of standardized data which are used by the program 110 in analysis to act as important measures of device integrity and functionality, as well as to note changes in underlying pathology of the patient's native anatomy.

These measures include (but are not limited to) the following:

1. Input pressure and flow rates.

2. Outgoing pressure and flow rates.

3. Flow directionality.

4. Flow velocity.

5. Viscosity.

6. Cellular composition (i.e., morphology, size, histology)

7. Structural integrity (i.e., thickness, porosity, diffusion, cross flow, defect size).

In addition to the program 110 having the ability to record and measure comprehensive device flow rates (i.e., from proximal to distal ends of the device), the ability to record focal measurements at the level of individual sensors provides an additional mechanism for identifying focal changes in flow velocity or directionality. As an example, suppose a small mural based abnormality (e.g., atherosclerotic plaque) occurs along the inner wall of device at its midpoint. The ability for locally situated sensors within the inner wall of the device to record focal velocity and directionality measures will provide the program 110 the ability to detect small localized changes in flow direction and velocity which may otherwise go undetected if one was to solely rely on “whole” device measures of velocity, pressure, and flow. This provides the program 110 with the ability to identify in three-dimensional space the exact location of the abnormal measurement, its degree of clinical/functional impairment, and the potential underlying etiology. Once the deficiency is identified, temporal data measurements can provide additional insight as to the progression of the abnormality, which can in turn assist in intervention. For example, a small localized deficiency in flow velocity which does not adversely affect “total velocity” and exhibits minimal change over time, can in all likelihood be conservatively managed with observation and continued surveillance, whereas a localized deficiency which adversely affects “total velocity” and substantially changes over time, may prompt more aggressive intervention. By the program 110 having the ability to reference database 113, 114 from a large pool of patients and devices, experiential insights can be gained to assist in establishment of “best practice” guidelines, specific to the patient and device profiles, along with the underlying data deficiency.

In addition to physical measurements (e.g., flow, direction, pressure), sensors embedded within the medical device can also contain the ability to record data specific to cellular composition. As an example, suppose the medical device in question now includes an esophageal stent, which has been placed in the setting of esophageal cancer to bypass an area of malignant obstruction. In addition to measuring stent patency at the proximal and distal ends of the stent, one must also be concerned about tumor extension along the body of the stent (which can traverse the outer and inner walls of the stent along its long axis). If sensors embedded within the stent wall possess the ability to track cellular composition of tissues which come in contact with the sensors, one could in theory monitor local tumor invasion. When malignant cells mare first detected on the outer surface of the stent and later extend to sensors on the stent inner surface, one knows that tumor has violated the stent walls and is beginning to encroach upon the stent lumen, which if not addressed, will lead to stent obstruction. In addition to quantifying the extent and rapidity of stent wall invasion by tumor, the sensors could also be provided with the ability to inhibit (or retard) tumor extension by the program 110 activating a sensor-derived physical response (e.g., heat, light, radiation, chemotherapy, etc.) aimed at local tumor destruction and maintenance of stent patency. This illustrates how the device embedded sensors can serve multiple functions through data collection and intervention.

Another unrelated example of sensor intervention may include the abdominal aortic stent graft which was found to exhibit focal leakage through the stent graft wall. In addition to the program 110 providing the ability to identify, localize, and quantify the severity of the leak, the involved sensors could also release a biocompatible compound (e.g., fibrin) which attempts to seal off the focal leak. The relative success or failure of this intervention can in turn be readily ascertained by successive sensor derived measurements.

In one embodiment, in addition to the program 110 recording, analyzing, and even intervening on abnormal data measurements related to the medical device and its surrounding milieu, the measures derived from each individual medical device can also be used by the program 110 to identify, characterize, and quantify pathology outside and part from the medical device itself. On a simplistic level, the pressure measurements derived at the proximal end of the medical device can provide valuable information regarding arterial inflow to the device. When this data is correlated by the program 110 with other arterial flow and pressure measurements in the same patient, one can indirectly identify external sources of pathology. In the example, of an arterial stent, if the arterial inflow pressure and velocity measurements are abnormal, the program 110 can identify that an obstruction is present proximal to the location of the medical device.

Now if one goes one step farther and the program 110 correlates this device inflow data with other synchronous device measurements (i.e., in the same patient and at the same time), the program 110 can use multi-device data to analyze pathology at different locations within the body. This highlights another important function of the present invention, which is the ability of the program 110 to correlate and cross reference data from multiple devices at the same time and in the same patient. Suppose, for example, the patient has four different arterial stents in the treatment of peripheral vascular disease. These stents are located in the abdominal aorta, right common iliac artery, right superficial femoral artery, and left common femoral artery. Sensor derived measurements from the stent in the right common iliac artery show the stent is patent and has no significant change in velocity or pressure across its length. However, when the arterial inflow data (i.e., sensors in the proximal stent) is correlated by the program 110 with data from the abdominal aortic stent (which is proximal to the common femoral artery stent), then the program 110 can detect there is a drop off in arterial pressure somewhere between the distal end of the abdominal aortic stent graft and the proximal end of the right common femoral arterial stent. The severity of this obstruction can be further identified by the program 110 based upon the degree of segmental pressure change between these two arterial stents.

In one embodiment, the program 110 compares the pressure and velocity measurements between the right and left common femoral arterial stents, and finds that the inflow measurements of the left common femoral artery stent are comparable to the pressure/flow outflow measurements of the abdominal aortic stent graft. These comparative device specific measures provide evidence that the obstruction occurs after (i.e., distal to) the aortic bifurcation and proximal to the right common femoral arterial stent, most likely at the origin of the right common femoral artery. If the obstruction had instead been located in the distal abdominal aorta (proximal to the aortic bifurcation), a comparable abnormality would have been expected in the left common femoral artery stent, which was not the case. At the same time, comparative pressure and flow inflow measurements conducted by the program 110, in the right superficial femoral artery stent, show no significant change in measurements when compared to the right common femoral artery stent, which would mitigate against an obstruction in the arterial segment separating these two stents.

In one important application of the present invention, the device related measurements can be sequentially analyzed by the program 110 to identify the timing, severity, location, and etiology of pathology. Using the same patient with 4 arterial stents (in the treatment of peripheral vascular disease), one can identify a sudden and rapid change in arterial inflow measurements in the right common femoral artery stent, accompanied by complete absence of distal stent outflow. This indicates that an acute obstruction has occurred in the right common femoral artery stent, the specific location of which can be determined by the program 110 analyzing neighboring sensor data along the course of the stent. The two likely causes of pathology are progression in atherosclerotic plaque or embolism. Since the “pre-event” measures showed a relatively mild degree of obstruction and the abnormity occurred quite acutely (i.e., in the 15 minute interval of routine sequential measurements), the logical etiology is that of embolism. Since the embolism source can occur anywhere proximal to the point of obstruction it is often difficult to localize the exact source. However in this case, analysis by the program 110 of the sensors in the internal wall of the abdominal aortic stent graft had previously demonstrated a significant burden of atherosclerotic plaque along the middle of the stent which is no longer detected. By measuring the distance between sensors and “before and after” sensor data, he program 110 can estimate the size of the embolus (i.e., 2.5 cm), which correlates with the luminal diameter of the occluded right common femoral artery stent. Knowing the etiology, source, timing, and severity of this obstruction can allow the program 110 to provide timely diagnosis, notification, and intervention. Having the ability of the program 110 to correlate real-time data from multiple individual devices, provides additional knowledge and insight not available when data is limited to that of a single medical device alone.

Using another example, suppose this same patient had an indwelling cardiac pacemaker due to an underlying cardiac arrhythmia. Analysis derived from the pacemaker sensors by the program 110, revealed a prolonged period of atrial fibrillation 24 hours prior to the event in question (i.e., embolic obstruction of the right common femoral artery stent). Since atrial fibrillation is a well-documented cause for cardiac thrombus formation and subsequent emboli, this could also serve as a source of the embolic disease. One method of differentiating between the two possible sources of emboli (i.e., cardiac versus abdominal aorta) is for the program 110 to analyze the flow data derived from the abdominal aortic stent graft data during the specific time frame of concern (i.e., the period of immediately preceding and up to the time the occlusion of the right common femoral artery stent was identified). If the thrombus had originated from the heart, then the embolus would have had to pass through the abdominal aortic stent before passing into and obstructing the right common arterial stent graft. This could have been identified by the program 110 by retrieving sensor derived data within the abdominal aortic stent graft during the time in question and evaluating for the presence of abnormal internal flow (e.g., loss of normal laminar flow, alteration in flow directionality, presence of an intraluminal mass separate from normal red blood cells). This last feature can be facilitated by the program 110 incorporating ultrasound capabilities within the sensors, which provides the ability to use ultrasound to analyze medical device internal flow and wall characteristics.

Sensor Analysis of In Vivo Physiology and Local Pathology

In one embodiment, the ability of medical device embedded sensors to provide real-time data for the program 110 to analyze medical device functionality and integrity, can be expanded to real-time analysis of patient physiology and local pathology. The ability of sensors to record and analyze data related to their surrounding milieu can also be extended to the evaluation of local tissue physiology and pathology. This ability to analyze adjacent cellular physiology and pathology can be accomplished in a variety of ways including (but not limited to) analysis of cellular morphology, histology, and chemical output. In addition to having nanotechnology directly embedded within the sensors for the program 110 to analyze adjacent cellular and tissue structure, chemical detectors can record data related to chemical compounds in local proximity to the sensors (e.g., infectious and neoplastic by products). In addition, sensors can also have integrated microscopic cameras, which provide a means by which in vivo photographic images can be obtained for external analysis by the program 110 and correlation with the sensor derived physiologic and chemical data.

A few examples of these applications include the following:

1. IUD placed in the endometrial cavity of the uterus for contraception.

2. Esophageal stent for treatment of malignancy

3. Orthopedic hardware placed in the spine for surgical fixation.

4. Vascular stent placed in an artery for treatment of vascular occlusion.

In the first example, the IUD is placed in the endometrial cavity of the uterus. Over time, if this IUD was to change position, this would be recognized by the spatial localization capabilities intrinsic to the device. In addition, if the sensors embedded in the external walls of the IUD had the intrinsic functionality to analyze cellular morphology and histology, the sensors could detect a change in local tissue composition as the IUD migrated from the endometrial cavity to the uterine wall (due the fact that cellular composition of endometrial and myometrial walls are distinctly different from one another). This cellular detection capability provided by the device embedded sensors would in essence, could have the program 110 provide an early warning sign to clinical providers that a change in IUD position has taken place which could serve as a precursor to uterine wall perforation. Upon recognition of this positional change in the device, clinical providers could elect to actively intervene by device removal or repositioning, or elect to increase surveillance in order to ensure that no further damage to the uterine wall was to take place.

In addition to the ability of analyzing adjacent tissue histology and morphology, the program 110 could also be used to detect changes in the local tissue and cellular environment related to pathology, such as malignancy or infection. If, for example, sensors in the IUD began to detect localized changes in the endometrium such as increased vascularity, changes in cellular composition (e.g., increased neutrophils), and chemical mediators (e.g., cytokines, histamine); that would have the program 110 provide an early warning sign of developing infection (i.e., endometritis), which would prompt device removal and/or antibiotic therapy. If the provider elected to initiate antibiotic therapy without device removal, the relative success of medical therapy could be locally monitored by having the same sensors measure the temporal response of these same infection markers (e.g., vascularity, neutrophils, and cytokines). This illustrates how device embedded sensors can be used to detect local pathology while also analyzing treatment response.

A similar analogy can be drawn for detection of malignancy. While it would be relatively rare for device embedded sensors to detect carcinoma in situ in an otherwise healthy patient, some devices may be placed in high-risk patients which could readily benefit from sensor detected local malignancy. As an example, suppose a patient with known esophageal cancer has had placement of an esophageal stent to maintain esophageal patency while receiving chemotherapy/radiation therapy. By having the ability of the program 110 to differentiate non-malignant from malignant cells in proximity to the stent walls, this could provide valuable information to the clinical providers regarding the relative success (or lack thereof) of cancer treatment as well as the risk for stent occlusion by invading malignant cells. If one was to expand the program 110 to perform a quantities time-activity analysis of malignant cell transformation in proximity to the stent walls, one could effectively estimate the time before stent occlusion would occur, which would be quite valuable in planning treatment.

By having the ability to integrate miniature cameras (or ultrasound transducers) within the sensors, one could take photographic or sonographic images in situ, which could provide visual evidence of local tissue characteristics. As an example, suppose a thrombus is beginning to form in the wall of a vascular stent. This would not only have the potential to lead to stent occlusion but could serve as a source of embolism (which could be life threatening). In the event that sensors embedded in the inner wall of the catheter detected local changes in cellular morphology as analyzed by the program 110, the program 110 could activate the camera or ultrasound functionality within these sensors to acquire photographic or sonographic images, which in turn could be transmitted via wireless technology for provider review. Serial images could assist the program 110 in quantifying the initial size and interval propagation of thrombus, which is critical for treatment planning. If, for example, the thrombus was detected at a very early stage (e.g., 4 mm in size), conservative treatment may be selected. Instead of treating with systemic anticoagulation therapy (which would be associated with high morbidity), an alternative local therapy could be employed in which a pharmacologic agent is locally released by sensors in direct proximity to the thrombus. This could result in improved clinical outcomes through combined early detection and treatment. Having the ability to continuously collect and analyze data from device embedded sensors would provide valuable data related to device surveillance, early detection of pathology, intervention, and analysis of treatment response. (Note the storage of pharmacologic agents within sensor compartments is another feature of the invention. An electronic signal could be transmitted using wireless technology. Once received and verified by the program 110, the specific compartment within the sensor with the pharmacologic agent of interest would then be opened by the program 110, resulting in release of the pharmacologic agent of interest. The specific pharmacologic agents stored within sensor compartments could be predefined based upon the type of device, anatomy in which it is deployed, and patient specific attributes. In the example provided, of an arterial stent in a patient with peripheral vascular disease, one of the commonly expected complications would be atherosclerotic plaque formation, which would in turn prompt inclusion of thrombolytic pharmacologic agents in the sensor storage compartments.)

One final example may include orthopedic hardware inserted for surgical stabilization of the spine. In addition to loss of device integrity and movement (which was previously discussed), another common complication is local infection, which can result in infection of the spine and requirement for surgical removal of the infected device. Conventional diagnostic options tend to result in delayed diagnosis and high degrees of patient morbidity. With the present invention however, many early signs of infection can be detected by the program 110 including (but not limited to) device motion, changes in local cellular composition (e.g., neutrophils), increased vascularity, and presence of chemical mediators (e.g., cytokines). Once again, the sensors provide the program 110 with the ability for early diagnosis, intervention, and analysis of treatment response.

Medical Device Disposal

One of the often forgotten (yet important) steps in the medical device usage life cycle is the last and final step of device disposal. The documentation of data related to this step is important in not only identifying the end of the medical device's life cycle but also important in preventing illicit repackaging (and reuse) of medical devices.

The data associated with this device disposal process includes the following:

1. Registration/identification of device

2. Registration/identification of patient

3. Identification/authorization of provider(s)

4. Geographic location (supporting GPS technology)

5. Date and time of disposal (beginning and ending times)

6. Chosen method of device disposal

7. Documentation of device destruction (embedded sensors)

8. Identification/authorization of witness

As in all previous steps relating to medical device usage, the initial steps in data collection require registration and identification of the device, providers, and patient. This ensures that all involved parties are accounted for and cannot be completed unless all data has been successfully recorded and verified by the program 110. The verification process requires the identification and authorization of an uninvolved third party, whose role is to ensure that the device disposal process and participating individuals are accurate and complete.

A date and time stamped record of the device disposal process is automatically recorded by the program 110 in the database 113, 114, which begins with the initiation of device disposal (as recorded by the provider of record) and ending with the documentation of device destruction. While conventional methods of disposal rely on human data input (which can be erroneous), the present invention utilizes the program's 110 identification of the destruction of the device embedded sensors for objective verification. A predefined set of sensors must be documented to be destroyed in order for the program 110 to initiate the disposal process, and for it to be verified and completed. These sensors are strategically localized at major functional locations in the device (e.g., inner and outer walls, proximal and distal ends), in order to ensure that critical device sensors have been effectively deactivated and destroyed. In order to ensure that external sensors (i.e., from other medical devices) are not illicitly used to mimic device destruction, upon entry of the device identification data and notification of device disposal, a predefined sensor map is presented by the program 110 to the provider which alerts them as to the specific sensors required for destruction and completion of the device disposal process. This can take the form of an electronic display on a display device 102, which highlights the predefined sensors earmarked for destruction by the program 110, or an implanted sensor activation system which has the program 110 emit a visual display of the specific sensors requiring destruction. One way this can work is that a wireless transmission is activated by the program 110 after device identification and input of the disposal command, which in turn will cause the predefined sensors to be visually activated (e.g., green light).

In one embodiment, in order for the sensor destruction to be documented and verified by the program 110, an external sensor destruction device is utilized, which has integrated GPS technology verifying its physical location, user identification technology (e.g., biometrics device, voice recognition software), and a device reader (which records the unique device identification data). There are two options for device disposal. In the first option, the entire device (e.g., vascular catheter) can be inserted into the disposal device which simultaneously destroys the designated sensors and entire device. In the second option, the designated sensors are removed (which effectively terminate device functionality) and disposed of by inserting into the disposal device. This option is more practical for larger more cumbersome medical devices (e.g., surgical hardware).

In the event that certain components of the device are deemed to be “reusable”, the disposal process has the program 110 recording these components at the time of device disposal and de-identifying them (since their identifying information was directly tied to the original device). These “de-identified” device components can then be transferred to the device manufacturer by electronic means by the program 110, where they can be reintegrated into new device technology and have new device identification incorporated, to identify the new device. Whenever a device component is recycled, all related data is maintained by the program 110 in the database 113, 114, which provides a mechanism for tracking the component over time. This provides a mechanism to enjoy quality control of each medical device in the event that recycled components are used.

In the event that an individual attempts to use removed device components without proper authorization or documentation from the program 110 database 113, 114, an automated “kill” option is activated by the program 110 within the device component, analogous to that of a stolen smart phone. This prevents illicit reconstruction of medical devices using unauthorized device components.

In some circumstances, a provider may inadvertently dispose of a medical device in an unauthorized fashion. In this situation a manual data entry of device disposal is required, which incorporates a number of extra data inputs (e.g., verification of device disposal by two unrelated individuals, sign off by the institutional compliance or quality assurance officer). In the event that the provider has repeated episodes of improper device disposal, remedial actions may be required by the program 110 (e.g., mandatory education/training programs, temporary loss of clinical privileges, requirements for device countersignature). As is the case for all database 113, 114 data, the device disposal data becomes an integral part of the provider profile, and in turn can be used by the program 110 in the evaluation of provider compliance, quality, and safety measures related to the medical device.

Medical Device Quality Control

Traditional quality control of medical devices is in large part left up to the individual end-user, and is predominantly limited to “high technology” instruments (e.g., CT scanner), using calibration tools. The vast majority of smaller medical devices (e.g., catheters) do not have standardized methods of quality control (QC), which most commonly includes periodic medical imaging tests (e.g., radiography) to ensure adequate placement.

As previously discussed, a number of tools, applications, and data measures are incorporated into the present invention, which provide for a standardized methodology for medical device quality control. In addition, embedded sensor technologies provide for a number of unique and standardized QC applications, some of which can be centralized so as to ensure consistency, accuracy, and reliability of the derived QC data. The sensors embedded within the medical device provide the ability for remote wireless access and data transmission. These sensors can be randomly tested to ensure functionality based upon a predetermined QC program or tested in response to a faulty data transmission. Since it is impractical to routinely test all embedded sensors within each individual medical device, the program 110 can mine collective and individual sensor-derived data from each medical device. In the event that this device-specific data is found to be incomplete or inaccurate, targeted testing of the device and its individual sensors can be performed, using the program 110 as required, in an attempt to verify sensor functionality and accuracy of the record sensor derived data.

If, for example, this external QC monitoring detected non-functioning of an individual sensor within a medical device, the device could in effect be “turned off” remotely by the program 110. This would prevent erroneous data from being collected from the malfunctioning sensor, which in turn could produce erroneous device-specific data. If, for any reason, the malfunctioning sensor was found to be restored to normal function, it could be reactivated by the program 110, which would provide for its derived data to be included in the overall device data analysis.

In one embodiment, if a critical number of sensors were found to be non-functional, thereby rendering the recorded device-specific data to no longer be valid and/or of practical use, an automated notification of “failed device QC” would be sent by the program 110 to the provider(s) of record. This would alert them of the data deficiency and provide them with the option for device removal and/or replacement. In some devices (e.g., vascular catheter for venous access), the inherent clinical functionality of the device would necessarily be compromised to require device replacement in the setting of non-functioning sensors. In other devices (e.g., cardiac pacemaker), the non-functioning sensors may compromise clinical functionality of the device to the point where device replacement is required. Other situations may be contextual in nature. Suppose for example, an intrauterine device (IUD) has been inserted into the endometrial cavity of the uterus for the purpose of contraception. Sensor derived positional data of the device reveals that the IUD has migrated from the central endometrial cavity to its periphery, juxtaposed to the uterine wall. As long as the IUD does not directly penetrate the uterine wall, it may still be clinically useful. If, however, further migration of the IUD was to occur, so that it begins to penetrate the uterine wall, it could become a danger for uterine wall perforation. As long as the sensors located in the portion of the IUD which is juxtaposed to the uterine wall are functional, continued surveillance of IUD position can be performed and the IUD left alone. If, however, the sensors located in the critical position of the IUD were found to be non-functional, then accurate positional assessment would be compromised. In this specific context, IUD removal would be prudent. This illustrates how sensor derived QC can be used to evaluate device safety and quality, while providing guidance to device maintenance and/or replacement.

Economic Ramifications

The data in database 113, 114, and derived analytics of the program 110, provide a number of compelling advantages for improving patient safety, security, quality, and clinical outcomes. As a result, it may be deemed advantageous (or even mandated) by third party payers that clinical providers utilize the invention when medical devices are being incorporated into the healthcare diagnostic or treatment plan. Economic incentives may be tied to a number of database 113, 114 deliverables including (but not limited to) device registration, provider/patient registration, device disposal, device quality control and quality assurance, and decision support applications (e.g., device selection).

Additional economic incentives can be created based upon database 113, 114 analytics being tied by the program 110 to device quality and safety measures including (but not limited to) device positioning, procedural time, adverse events, functionality, longevity, and follow up care requirements.

Sensor derived data is an important component of the present invention which relies on the accuracy of the sensors embedded within the medical device to record accurate and reproducible data into the database 113, 114 in a standardized format. If the sensors are not properly functioning, the program 110 derived data will be inaccurate and undermine the utility of the data being recorded. In order to ensure that the sensors are accurately functioning, each sensor contains a microchip which can be remotely accessed for external quality control testing. In the event that the sensor is determined to be non-functional, then all subsequent data associated with that individual sensor will be discarded and not included in any subsequent analyses. If sensor functionality can be remotely restored, then the derived sensor data will be continued to be used.

Since the data derived from each individual sensor can be correlated with comparable data from adjacent and parallel sensors (i.e., next to or opposite to) within the medical device, then any time the program 110 does not correlate the data with its “comparable” sensors, the program 110 would provide an automated alert that either a pathologic state is present, or specific sensor data is inaccurate. In addition to requesting repeat sensor data acquisition (to verify the accuracy of the originally recorded abnormal data), a QC request can be transmitted to activate the sensor QC testing procedure, with a focus on the sensors in the specific location of interest.

Since sensor functioning and reliability is an intrinsic component of the medical device and derived data analytics, sensor quality control (QC) is included in the program's 110 overall analysis of medical device performance and functionality. Repeated sensor QC deficits is in itself a source of medical device deficiency and is included by the program 110 in the overall medical device performance analytics.

In one embodiment, the creation of the database 113, 114 also provides for the program's 110 creation of automated authorization of the medical device and procedure using neural networks and other artificial intelligence techniques based upon established best clinical practice guidelines. In the event that the proposed procedure and/or medical device selected are determined by the program 110 to be inconsistent with these established guidelines and rules, the clinical provider can request an exception to be granted (through direct communication with established clinical experts), or modify the procedure/device in keeping with the established standards. The program 110 neural network derived recommendations can include a number of acceptable procedural and/or device alternatives which can be chosen by the clinical provider in an effort to expedite the procedure and forgo a formal (and often time consuming appeal). This ability to create a computer-derived hierarchical analysis of “best practice” procedures and devices is another important and unique feature of the present invention.

In one embodiment, once the procedure and device have undergone the computerized authorization process, the provider's provider (including historical procedure and device data) can be cross referenced by the program 110 to determine how their utilization compares with peers. In the event that a significant statistical outlier is identified (e.g., excessive use of an individual medical device which is not deemed to be consistent with best practice), an automated quality assurance (QA) alert can be activated by the program 110 which in turn will trigger further more in depth analysis by clinical experts in that particular medical specialty. The primary purpose for this provider utilization analysis by the program 110 is twofold: firstly, identify fraud in terms of overutilization, and secondly, to assist in provider education of best practice guidelines and use of the database 113, 114 for decision support.

In one embodiment, after the program 110 procedure has been successfully completed (with a number of intervening steps which are described in detail below), the procedural data input process is completed. At this time, an automated billing and reimbursement process can be initiated by the program 110 which directs payment to the institutional and individual providers in accordance with the procedure performed, clinical diagnosis, and medical device(s) used. In addition to the baseline payment, the program 110 also creates the ability to perform comparative analysis, which can be used for supplemental incentive payments based upon established safety and quality metrics.

The ultimate goal is to improve medical device related clinical outcomes through active participation and utilization of the database 113, 114 and its derived analytics. If financial incentives can be created which tie performance (i.e., improved quality and safety) with reimbursement (i.e., Pay for Performance), then improved clinical outcomes may be achievable. The present invention provides a methodology for creating such a Pay for Performance system relating to medical devices.

Ongoing Safety and Quality Surveillance

One of the major deficiencies in existing healthcare practice is the lack of feedback provided to regulatory agencies and other healthcare providers relating to medical device safety and quality after it has been introduced into the marketplace. While the FDA approval process is fairly rigorous and requires exhaustive data collection and analysis to ensure the proposed medical device will be safe and clinically effective relative to its intended use, ongoing data once the device has been introduced into the market is relatively sparse and is largely retrospective in nature. This often results in delays in identifying quality and safety concerns, as was the case for uterine morcellators, which were recently found to be associated with higher than expected rates of uterine cancer. Had the program 110 been employed, prospective data would have been continuously collected and analyzed, thereby providing healthcare researchers, clinical providers, and governmental agencies with improved insights related to medical device safety and performance. Even before statistical evidence for increased cancer risk is identified by the program 110, the negative performance and/or quality data recorded in the database 113, 114 could provide important feedback and decision support for clinical providers in device/procedure selection and treatment planning. In addition, early data outliers may serve as a catalyst for governmental regulatory agencies to increase surveillance and data collection to those devices with unexpectedly poor quality and performance metrics, while simultaneously specific patient profile groups at increased risk for diminished safety or performance, relating to specific medical devices.

The following provides one embodiment of the method of the present invention.

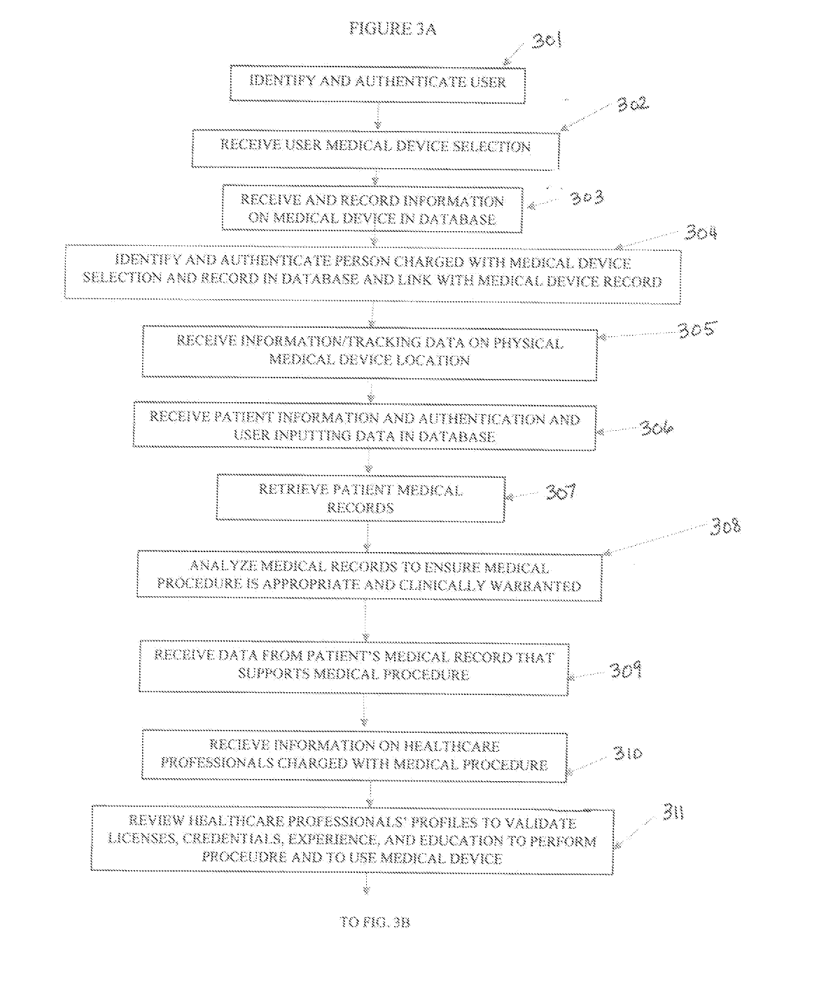

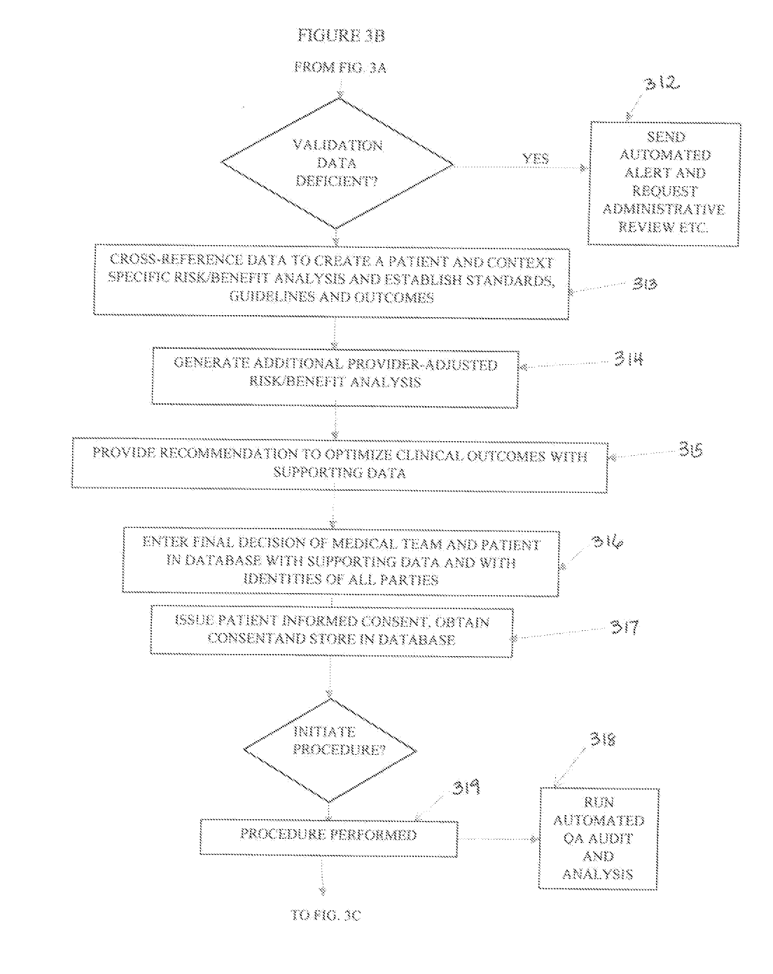

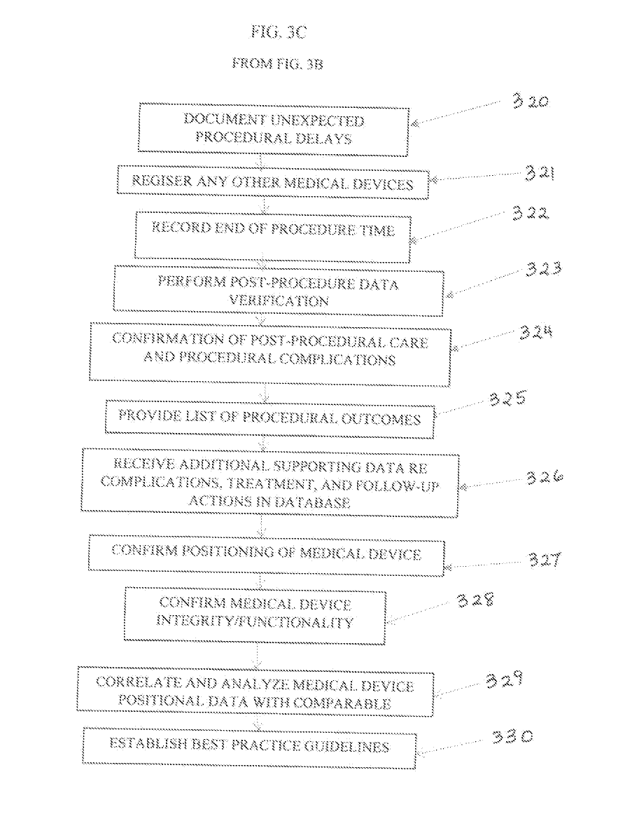

In FIG. 3A, step 301, the program 110 receives login input from a user, and other identification information (i.e., unique identifier, biometrics, RFID etc.), in order to authenticate the user and provide access to the database 113, 114, or medical device inventory (optional), which information is recorded into the centralized database 113, 114.

In step 302, the program 110 receives the user's election of medical device(s) of interest.

In step 303, the program 110 receives information on the medical device, which information may be captured by a scanning device, for example, and which identifies the medical device and records the information into database 113, 114. Thus, the program 110 can record and track usage and ancillary clinical, technical, personnel, and analytical data associated with the specific device. This medical device identification process involves both identification data embedded in the external packaging and the device itself, which is stored in the database 113, 114. The downloading of this data is routinely performed in an automated fashion through the use of an external data transmission device (e.g., RFID, electronic scanner, etc.).

In step 304, the program 110 receives identification and authentication of the individual(s) tasked with device selection and preparation, with data recorded in the database 113, 114 and directly linked by the program 110 to the medical device(s) of record.

In step 305, the program 110 receives information on the physical location of the medical device. In some cases, transport of the device to the physical location in which the procedure is to be performed may be required, and recording of this physical location into the database 113, 114 is performed. A default institutional location is automatically presented by the program 110 based upon the medical device storage and purchasing data. If the final location of usage is different from the default location, the alternative data can be manually input, along with the identity of the individual responsible for location input. (In another embodiment, an optional GPS tracking device can also be embedded in the device for automated location input and/or confirmation into the database 113, 114 by wireless means. In yet another embodiment, internal tracking systems (e.g., using RFID or GPS technologies) may be created within healthcare institutions which can identify the exact location of medical events and personnel. When used, these internal tracking systems would provide an alternative method for computerized location tracking and automated recording of locational data by the program 110.)

In step 306, the program 110 receives inputs regarding the patient identification and authentication, and the input is stored in the database 113, 114. The data on patient identification can be obtained using a variety of technologies (e.g. RFID, biometrics, wristbands) and requires human confirmation prior to performance of a medical procedure. The identity of the healthcare professional tasked with patient identification is also recorded into the database 113, 114 by the program 110 along with the date and time of patient identification/authentication.

In step 307, once the patient has been identified and authenticated, the patient's medical records are automatically retrieved by the program 110, from the database 113, 114, and an analysis performed by the program 110 in step 308, to ensure that the planned medical procedure is appropriate and clinically warranted. The data sources used for this process of procedural validation include (but are not limited to) the Patient Profile, physician orders, consultation notes, history and physical, laboratory data, medical imaging reports, and progress notes. In addition to manual data input, the program 110 can perform computerized data mining and utilize artificial intelligence technologies (e.g., natural language processing, neural networks) in the process of data review, extraction, and analysis. The specific data from the patient's medical record obtained in step 307, which is used to justify the planned procedure, are subsequently entered into the database 113, 114 in step 309 after the analysis of step 308.

In step 310, the physician/s (and/or alternative healthcare professional) who are tasked with performing and/or supervising the planned medical procedure are formally identified and their information is authenticated by the program 110 and stored in the database 113, 114.

In step 311, once the identities of all relevant healthcare professionals have been completed and entered into the database 113, 114 in step 310, the program 110 will undertake a review of each healthcare professional Provider's Profile, in order to validate they have the appropriate licenses, credentials, clinical experience, and educational requirements required to perform the planned procedure, and to use the selected medical device. If any requisite data is deficient (see FIG. 3B), the program 110 will send an automated alert in step 312, notifying the involved parties, and request an administrative review prior to proceeding. In the event that this administrative review does not successfully clarify and rectify the stated deficiency, the procedure and usage of the device are deemed to be “unauthorized”. This will result in failure to authorize billing and reimbursement, as well as mandate an official institutional and provider quality assurance review.

In step 313, the combined medical data, planned medical procedure, and medical device information are then cross-referenced by the program 110 with the centralized database 113, 114 to create a computerized Patient and Context Specific Risk/Benefit Analysis. This analysis utilizes established best practice guidelines, evidence-based medicine (EBM) standards, and clinical outcomes data of large patient populations with comparable clinical attributes and patient profiles.

In step 314, an additional Provider-Adjusted Risk/Benefit Analysis can be performed which takes into account the performance metrics, clinical experience, and education/training data specific to the providers who have been identified as performing and/or supervising the planned procedure and medical device usage.

In step 315, based upon these individual or combined Risk/Benefit Procedural and Medical Device Analyses, the program 110 will provide a recommendation in an attempt to optimize clinical outcomes based upon the available data and established practice standards. Alternative recommendations could include alteration of the planned medical procedure, replacement of the medical device, or referral to an alternative healthcare provider (which may be on an institutional and/or individual provider level). The recommendations along with supporting data from the database 113, 114 are presented by the program 110 for review and consideration by both the medical team and patient.

In step 316, once a final decision is arrived at by the medical team, with the approval of the patient, this decision is entered into the database 113, 114 along with supporting data confirming the final decision, and the identities of all parties included in the decision making process.

In step 317, once the medical procedure and medical device have been finalized, the patient informed consent is issued, obtained from the patient, and the approved consent is entered into the database 113, 114. The patient and context specific risk/benefit analysis (and corresponding data) are included in the informed consent along with alternative options relative to the medical device being used and the procedure to be performed. In the event that the procedure and/or device are contrary to the program 110 data-derived recommendations, an explanation of the decision making process is directly incorporated into the Informed Consent document and requires signatures of both the patient (or legal guardian) and alternative healthcare provider (e.g., patient's primary care physician, hospital chief of staff, department chief).

The next step is Procedure Initiation, which can only take place after successful and complete registration of the medical device, patient, and providers, along with computerized program 110 analysis of the Risk/Benefit Analysis. If a procedure is attempted to be performed without completion of these events, an automated QA audit and analysis will be triggered by the program 110 in step 318, which can be automatically integrated with an escalation pathway if the procedure being performed or Risk/Benefit Analysis fulfills predefined criteria for emergent status. In the escalation pathway, a predefined schema is followed which mandates alert, receipt acknowledgement, and follow up action by designated individuals within the institutional provider (e.g., compliance officer, department chief, hospital administrator). The resulting information is recorded in the database 113, 114 by the program 110, for future analysis and intervention (e.g., administrative oversight, formal review by an accredited organization (e.g., CMS, Joint Commission)). In addition, any unresolved medical device registration and approval process can be directly linked by the program 110 to third party financial reimbursement (e.g., CMS, private insurance plans), so that an unresolved procedure will not be reimbursed, and the offending parties may be subject to disciplinary review and/or loss of billing credentials within the payment network.

In step 319, once all data entry and analysis has been completed and the planned procedure, medical device, and providers are deemed to be “clinically acceptable” and consistent with “established practice guidelines”, the procedure can be performed. This will entail re-registering all procedural participants and medical devices, along with a formal acknowledgment of “Procedure Initiation”, which in effect results in the date/time posted as the “Procedural Start Time”. If this step is overlooked, the default start time will be recorded by the program 110 in the database 113, 114 as the time immediately following successful completion of device, patient, and provider registration and the Risk/Benefit Analysis. Failure to complete this Procedure Initiation step will adversely affect procedural and individual provider analyses by prolonging the “procedure performance time”, which is an important metric used in performance assessment and comparative analyses.

After the procedure is initiated, any number of potential delays or unforeseen events may take place which unexpectedly prolong the procedure time and/or adversely affect technical/clinical success of the procedure (e.g., breakage or other technical failure of the medical device, deterioration in patient clinical status).

These can be documented by the program 110 as “unexpected procedural delays” in the database 113, 114 in step 320 (see FIG. 3C), along with the corresponding duration of the delay. Since these are unconfirmed and subject to manual data input (since no one can accurately document when the delay actually began), they will be treated as “unconfirmed data”, which is recorded in the database 113, 114 by the program 110, but not directly incorporated into formal statistical analysis. These delays can however be included in a separate category of “Unconfirmed Procedural Delays”, which serve as a means of documenting delays, causative factors, and estimated time impacts on procedural outcomes.

During the course of performing a procedure it is not uncommon for more than one medical device to be used. This could be due to a variety of reasons (e.g., wrong size, breakage) and requires use of a second (or third) device, which requires registration for appropriate documentation and accurate data analysis in the database 113, 114 by the program 110 in step 321. If this second device was not registered, it would result in both inaccurate longitudinal analysis and failure to identify the device in the future. In order to ensure that these additional medical devices are accurately recorded in the database 113, 114, an expedited “in procedure” device registration process is made available by the program 110, which provides for associated registration data from the original device to be automatically populated into the new device registration process. As an example, the default registration data attributable to the patient, procedure being performed, and providers are assumed to be the same as that recorded for the original medical device, and are therefore auto-populated in the new device registration database 113, 114. If any of these variables have changed, a provider in a supervisory role (e.g., surgeon, nursing supervisor) would have to re-register (in order to identify the person responsible for new data entry), and then modify the data input related to the change in question. An example of such a modification might include a device associated with a procedural complication (e.g., pneumothorax (i.e., collapsed lung) during the course of a lung biopsy necessitating insertion of a chest tube), or change in the procedure being performed (e.g., change in intravenous catheter insertion from right femoral vein to left subclavian vein due to injury to the right femoral vein and removal of the original venous catheter). The expedited medical device registration allows for rapid registration while also ensuring that all relevant and updated data is accurate. As the new device is registered, an amended time stamp is recorded by the program 110 in the database 113, 114 which identifies the new start time for the second medical device, which runs concurrently with the original start time (from the first medical device) to record “total procedure time” and “secondary procedure time” independently.

In step 322, once the procedure has been completed, the end time of the procedure is recorded by the program 110 in the database 113, 114. It is important that the recorded “procedure end time” be accurate and truly represented by absolute completion of the procedure, since there is a benefit to be derived from the providers to artificially reduce the “total procedure time”, so as to improve their personal performance time statistics. A number of methods can be employed to accomplish this goal which include (but are not limited to) the requirement for all involved parties formally register procedure completion, have an independent observer (e.g., nurse supervisor) document procedural completion, track patient location (e.g., using RFID wristband, for example) to denote the time in which patient transfer took place, time stamped event tied to physician post-procedure orders in the patient's EMR, and/or incorporate an additional step for procedural cleanup in which a formal time stamped event takes place tied to the registration of a sterilizing agent, device, or third party.

In step 323, after completion of the procedure, all recorded data must be formally validated by the program 110 and amended, if necessary. The party responsible for completing this “Post-Procedure Data Verification” step is the provider overseeing and in charge of the procedure performed. In this step, the provider would re-register (having already completed the identification/authentication process) prior to the procedure being performed) and select the option for “Post-Procedure Data Verification”. All recorded data would be presented by the program 110 to the provider for verification at that time, which may be displayed on a timeline which tracks all sequential registered events. In the event that some data point was missing or determined to be erroneous by program 110 analysis, the provider could simply select the “Edit Timeline” option and insert and/or edit the missing and/or erroneous data, along with accompanying information for explanation. If this editing mode is utilized, an additional confirmation step is required by the program 110 to ensure data accuracy. This would require the registration of a second individual who was previously registered as a participant in the procedure, who would then confirm the accuracy of the edited data. The identities of these individuals and edited data would be recorded by the program 110 in the database 113, 114 for future analysis. In addition, all “Post-Procedure Data Verification” events associated with data editing would be flagged by the program 110 and separately analyzed by the program 110 and quality assurance (QA) personnel to ensure data accuracy, potential for equipment malfunction (e.g., database not automatically capturing requisite data), and the frequency of editing. If an individual provider, medical device, or procedure was associated with a higher frequency of data editing this may serve as a trigger for more in depth review by the program 110.

In step 324, confirmation of two additional important components of data recorded in the database 113, 114 by the program 110, during the “Post-Procedure Data Verification” step, are Procedural Complications and Post-Procedural Care. These are important because they form the basis for post-procedural outcomes analysis (which is an extremely important metric used in program 110 analysis of quality, safety, economics, and provider/device performance.

In the recording of Procedural Complications, the primary provider is required to input any and all data into the database 113, 114, which is deemed attributable to the performance of the procedure which may have a negative impact on clinical outcomes and patient care.

In step 325, a standardized list of Procedural Outcomes is presented by the program 110 to the provider, which provides a method for recording standardized data in the database 113, 114 for analysis by the program 110. A representative list of standardized options includes the following:

a. No procedural complication

b. Minor procedural complication (no clinical treatment of follow-up required)

c. Major procedural complication (minor clinical treatment and/or short term follow-up required)

d. Severe procedural complication (major clinical treatment and/or intermediate to long term follow up required)

e. Catastrophic complication (patient death or chronic debilitation)

In step 326, whenever the provider inputs options b-e, additional supporting data is required by the program 110 which describes the nature of the complication, resulting clinical treatment, and follow-up actions taken. These data are subsequently recorded by the program 110 in the database 113, 114 for future analysis.

The recording of “Post-Procedural Care” can be divided into two broad categories: routine and non-routine post-procedural care. Routine care is that which is customary for the procedure being performed and expected for all patients undergoing the procedure of record. Non-routine care may include care provided for one of two principle reasons: either a procedural complication has taken place requiring additional (and unexpected) intervention, or the patient's clinical status requires additional care above and beyond that of most patients undergoing a similar type of procedure. This latter case is often associated with higher morbidity patients, which may be the result of comorbidities and/or advanced disease states. Classification of these “higher risk” patients is typically identified by the healthcare professional prior to the planned procedure and recorded in the Patient Profile in the form of a standardized Patient Morbidity Score. This provides a method for classifying patients undergoing a specific medical procedure into groups of “similarity”, which in turn will provide valuable data for longitudinal statistical analysis by the program 110 regarding Risk/Benefit Analysis and Best Practice Guidelines (specific to patient profiles, disease states, procedures, and medical devices).

The data associated with “Post-Procedural Care” can include a number of clinical treatment and diagnostic options and include (but are not limited to) clinical consultations, imaging studies, laboratory tests, medications, therapeutic regimens, and preventive care. These are routinely contained within the Physician Orders section of the patient EMR and can be electronically linked or downloaded into the database 113, 114 by the program 110. In the event that a patient had a severe or catastrophic complication, sequential analysis of the Physician Orders may be required to capture all relevant Post-Procedural Care data. Since this presents a logistical and resource intensive challenge, this long term data collection may be limited to those orders which occur within a limited time frame to the procedure (e.g., 24-48 hours). The primary goal however is not to capture all post-procedural order, but instead to create a standardized and reproducible method for classifying post procedural follow up requirements as they relate to patient profile, performed procedure, device, and iatrogenic complications.

Now that the procedure has been completed and all procedural-related data have been recorded and characterized, the subsequent steps in data recording and analysis by the program 110 relate to disease surveillance, treatment, and integrity/functionality of the medical device. From a technical standpoint, it is first important in step 327, to confirm the correct positioning of the medical device in question, followed by adequate functionality. If for any reason either of these factors are incorrect, some sort of remedial action is required, depending upon the specific type of device and severity of the problem. As an example, a “low technology” medical device such as a feeding tube in a suboptimal position (e.g., distal esophagus), may merely require advancement for optimal positioning before use. Once successful advancement has taken place and been documented (e.g., using radiography), the device can now be operational. On the other hand, a more complex medical device (e.g., orthopedic hardware) which is incorrectly positioned may require repeat surgery, in order to remove the malpositioned hardware and reinsert new hardware. Since device positioning is an integral component to patient safety, functionality, and long-term clinical viability it is imperative that this first post-procedural step of positioning is objectively documented and verified.

In order to accomplish this, a series of documented metrics must be recorded in the database 113, 114, prior to clinical use of the device, which include the following:

a. Medical device attributes (prepopulated from procedure data contained within the database 113, 114)

b. Date and time of device placement completion (i.e., end time of procedure)

c. Identity of provider/s responsible for device placement

d. Method for assessment of device positioning

e. Identity of responsible party for reviewing and analyzing positioning data

f. Standardized results of positioning data, including:
i. Grade 1: Device in optimal anatomic position, no adjustment or follow-up required
ii. Grade 2: Device in anatomic (but slightly suboptimal) position but requires no adjustment for routine clinical use
iii. Grade 3: Device in suboptimal anatomic position, may be clinically used but requires follow up for surveillance and verification of functionality
iv. Grade 4: Device in suboptimal position, requires documented repositioning prior to use
v. Grade 5: Device in suboptimal position, requiring immediate removal*
*Grade 5 device positioning requires critical results communication (i.e., via escalation pathway) by the program 110 at the time of determination, which must be documented in the database 113, 114 and patient EMR. An automated escalation pathway is incorporated to ensure communication is completed in a predefine time period. All forms of communication are recorded by the program 110, including the identities of involved parties, time stamps, and follow up actions taken.
   1. Non-emergent removal required
   2. Emergent removal required

g. Follow-up requirements (if applicable)

h. Date and time of positioning data completion

In step 328, once the device positioning has been verified and determined by the program 110 to be sufficient for use, device integrity/functionality is assessed. Determination of device integrity and functionality is obviously a variable matter, specific to different types of devices. As an example, if one wishes to assess functionality and integrity of an intravenous catheter, they can perform a radiograph to document the catheter is intact (i.e., no sheared catheter fragment) and aspirate blood to verify patency of the catheter. A more sophisticated type of intravenous catheter, such as a Swan-Ganz (SG) catheter would routinely require additional testing to ensure functionality, which in this case includes pulmonary artery blood pressure measurements. Once the blood pressure measurement recordings are deemed to be accurate (and the catheter positioning has been verified), it is now accessible for clinical use. In either case, any change in the functional status of the intravenous catheter will require some sort of clinical assessment to determine whether additional action is required. For example, if attempts to aspirate blood from the intravenous catheter are no longer successful, simple repositioning of the catheter may be required. Alternatively, if the SG catheter is no longer providing adequate pulmonary artery pressure measurements, a chest radiograph may be required to reassess catheter positioning prior to any intervention.

While assessment of device functionality is often tied to its positioning (as in the case of the aforementioned intravenous and SG catheters), functionality can also be intrinsically related to the internal components of the device itself. Medical devices which rely on intrinsic electrical components (e.g., cardiac pacemaker, neuromuscular stimulation device) may be properly positioned but fail to work properly. In the example of the cardiac pacemaker, lack of functionality could be catastrophic to the patient, who could die of a cardiac arrhythmia if the pacemaker is non-functional. As a result, routine device quality assurance (QA) testing is required for ensure functionality and this QA data should be recorded in the database 113, 114 for the purpose of longitudinal performance assessment.

Often times, subtle change in device positioning can serve as a precursor or warning to impending loss of device integrity and/or functionality. As an example, a small shift in positioning of orthopedic hardware (e.g., hip prosthesis, spinal pedicle screws) may serve as an early warning sign of device loosening and/or breakage. In extreme cases (e.g., aneurysm clip in the brain), the slightest movement of only a few millimeters can be potentially catastrophic, since this could be a warning sign of impending intracranial hemorrhage. For these reasons, objective analysis of medical device positional change is an important component relating to patient safety and functionality. In conventional medical practice, assessment of device positioning is largely idiosyncratic in nature. The most common method in which medical device positioning is evaluated is through medical imaging exams (e.g., radiography, CT). While this method of evaluation will certainly demonstrate gross positional change, it will often fail to detect subtle positional change over time due to a variety of reasons including (but not limited to) technical variability (e.g., patient positioning), lack of a standard anatomic positioning reference (i.e., specific to the medical device), interpretation error (i.e., physician analyzing the medical images), or inefficient communication (i.e., between the radiologist and physician responsible for the medical device). The net result is that subtle medical device positional changes frequently go undetected, which may adversely affect patient care and clinical outcomes.

The present invention addresses the current deficiency in medical device positioning assessment by having the program 110 integrate a variety of new technologies which objectively analyze device positioning, detect subtle positional change over time, and automatically record all measurements in the database 113, 114. The measurements are incorporated into a critical results communication pathway by the program 110 (in accordance with the extreme importance of positional change for each individual medical device, clinical setting, and individual patient).

In one embodiment, the device positioning tool utilizes two different options. In the first option, anatomic markers are positioned in adjacent fixed anatomic structures at the time of medical device placement. This option is optimal in the setting of surgical hardware, which are not expected to move in relationship to surrounding anatomy. The medical device is situated with a series of small anatomic sensors which can be distributed throughout the surface of the device, which in effect creates a three-dimensional record of the device location in vivo. At the same time, similar sensors are embedded in adjacent fixed anatomic structures, thereby providing a three-dimensional reference point of device positioning relative to adjacent anatomy. Since the reference anatomic markers are embedded within fixed anatomy which does not move, any subsequent positional change between the device and anatomic markers is assumed to represent positional change of the device.

An example of how this could be used is with an orthopedic device (e.g., hip prosthesis), which has a series of sensors on the prosthesis surface, which can be mapped to a computer generated prosthesis map. Similar sensors are embedded in fixed surrounding structures (e.g., femoral shaft, cortex, and acetabulum) at the time of surgery. The combination of these sensors will in effect create an anatomic map of device positioning relative to surrounding anatomic structures. During patient activity, any slight positional changes in these two sets of sensors (i.e., device and surrounding anatomy) will be recorded and serve as the baseline for exercise induced positional change. In some situations, these baseline positional changes can be recorded relative to specific activities (e.g., hip flexion, hip rotation) in order to identify how small “expected” positional changes occur relative to the specific activity of record. In addition, these positional sensors can also serve as a diagnostic guide for stress induced activity (e.g., weight lifting, running) to assess how device movement changes in the course of specific stress inducers.

If, however, a recording of positional change exceeds the established baseline measurement, then one would be concerned for pathologic positional change. As an example, a hip prosthesis routinely has small positional changes of 1-2 mm, which are most pronounced during strenuous activity with hip flexion (e.g., running). All of the sudden, positional change measurements for 4-5 mm are recorded which coincide with the same activity which previously was associated with only 0-2 mm of device positional change. This would alert the orthopedic surgeon to the possibility of prosthesis movement during running, which in effect represents prosthesis loosening. The recorded data for device position can also undergo time-activity curve analysis by the program 110, which records positional changes over time, tracks longitudinal change, and allows correlation with activity. This analysis will allow the clinical provider to determine the severity of the problem at hand, inciting events, and the response to intervention. As an example, the orthopedic surgeon might attempt physical manipulation or physical therapy of the involved hip in an attempt to improve flexibility and limit prosthesis movement with stress. The time-activity curve analysis of device position would provide an objective measure of positional change pre and post intervention, in order to assess intervention success and the need for additional intervention.

In step 329, the ability to correlate and analyze this device positional data with those of “comparable” patients and medical devices (using the Medical Device and Patient Profiles) provides additional guidance to the clinical provider and patient in determining best clinical practice guidelines and treatment options in context with the clinical condition, specific patient attributes, and technical measures intrinsic to the specific medical device. As an example, if the patient with the aforementioned hip prosthesis has a higher than normal level of everyday activity (e.g., competitive runner), then one would have a lower threshold of intervention when compared with a patient with a lower level of everyday activity, who is placing less stress on the involved hip. At the same time, suppose this patient has a larger body habitus (e.g., 72-inch height, 220 pounds); which further causes increased stress on the involved hip prosthesis. Comparative analysis by the program 110 of the Patient Profile database 113, 114 should take into consideration both variables (i.e., level of activity, patient body habitus) in order to determine optimal treatment strategy. At the same time, the specific type of medical device is also of primary concern, since different medical devices would be expected to behave differently in response to physical stressors. This use of the program 110 to analyze the comprehensive database 113, 114 (including Patient and Device Profiles), to analyze device positional data, provides an objective data-driven method of optimizing medical device selection, surveillance, and treatment.

Another unique method of analyzing medical device position in vivo is through the use of blood borne sensors (e.g., nanobots) which can be injected into the blood stream (or other circulating anatomic fluids like cerebrospinal fluid).

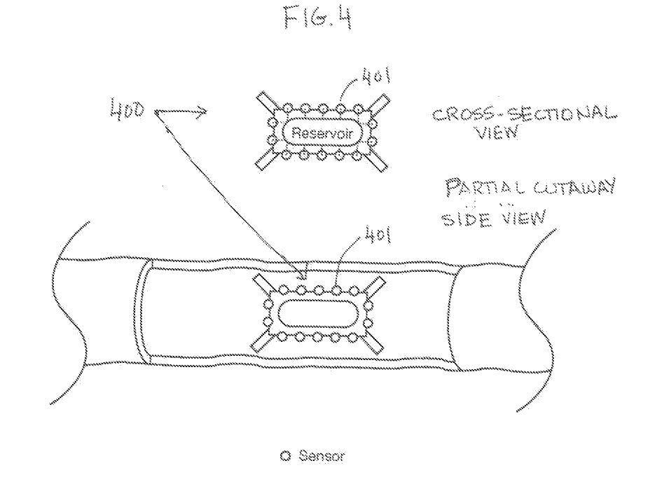

In one embodiment, the nanobot medical device 400 (see FIG. 4) can contain embedded biosensors 401 which are used for “mobile” diagnostic and therapeutic applications. In essence, the biosensor/nanobot 400 is a self-propelled, fully functional medical device 400 which has the ability to travel throughout an organ system and interact with local tissue or other medical devices.

In one embodiment, the migratory nanobot 400 contains high concentrations of embedded diagnostic biosensors 401 which can be customized specific to the organ system in which it is deployed (e.g., bloodstream, cerebrospinal fluid, gastrointestinal tract, genitourinary tract, etc.). During the course of the nanobot's 400 travels, it may continuously or periodically obtain local cellular or fluid specimens. When focal pathology is detected, the specific anatomic region of concern can be localized (i.e., marked) by deploying a biologic marker (e.g., diode, radiotransmitter, etc.), which can serve as a marker for future localization and intervention.

Once these circulating biosensors are introduced into the body that continuously emit a signal which can be tracked relative to its anatomic location in the body, as well as its position relative to other fixed biosensors. In the previous example of a hip prosthesis which has embedded biosensors, the circulating biosensors could both emit and receive signals from the fixed biosensors of the medical device, thereby providing an objective method of detecting biosensor positioning. This data can be transmitted via wireless technology to an electronic device which can record the corresponding data of the circulating and fixed biosensors. This would not only provide an accurate record of device positioning at a single point in time (i.e., static positional data), but also (and more importantly) provide a temporal record of device position (i.e., dynamic positional data). Since individual measures may be prone to slight variability (e.g., position of the circulating biosensor) within the blood vessel lumen), one can mathematically determine the standard deviation of biosensor positional measurements. When new positional measurements consistently exceed baseline measures, the program 110 would provide an alert to the possibility of device positional change. As successive measures are recorded, a clear and unequivocal picture would evolve as to device position over time (allowing for the established standard deviation of positional data).

This ability to objectively determine device position using mobile (i.e., nanobots) and stationary (i.e., device) biosensors is to some degree affected by the proximity of these two sets of biosensors. One would conjecture that the closer they approximate one another, the more accurate the subsequent measures. As an example, a device situated either within or in close proximity to a blood vessel would be preferable of this method, especially when extremely small degrees of device positional change are of high clinical significance. Devices such as vascular stents, aneurysm clips, and intravascular filters would be ideally suited for such a technology. In other anatomic milieus (e.g., central nervous system with cerebrospinal fluid (CSF)), medical devices such as intraventricular shunt catheters or neuromuscular stimulation devices could be analyzed for positional change using injectable nanobots which circulate within the CSF. The degree of “acceptable” device positional change will vary in accordance with the specific device and its anatomic location. Surgical clips in association with an aortic-femoral bypass graft may accommodate small optional changes of 2-3 mm, whereas comparable positional change of 2-3 mm in association with a brain aneurysm clip could be life threatening.

In addition to device positioning, another feature of the present invention is the ability to assess permeability and integrity of the device wall and/or external structure. By embedding biosensors in outer layer or external structure of the device, one can monitor device integrity by continuous measurements of the surrounding physical environment in which the medical device is situated. If the device is situated within a vascular lumen (e.g., endovascular stent graft), blood flow within the stent graft should be intraluminal only. In the event that the walls of the stent graft are compromised in any way, blood flow may now occur both inside (i.e., intraluminal) and outside of the graft (i.e., extraluminal). In addition to determining the loss of device wall integrity, the biosensors can also determine the severity of the integrity loss through volumetric and/or pressure measurements over time. This temporal analysis of device integrity can be essential to determining the type and timeliness of intervention. As an example, an endovascular stent graft used for the treatment of an abdominal aortic aneurysm would be expected to contain all blood flow passing through the length of the stent graft, which corresponds to the aneurysm. In a normally functioning stent graft with intact walls, all flow would be intraluminal in nature and exhibit laminar flow characteristics. If, however, the stent wall became minimally compromised (which may be beyond detection on conventional imaging studies like CT angiography or ultrasound), subtle changes in both internal flow characteristics and wall integrity may be recorded. Over time if undetected, the associated stent graft defect would be expected to enlarge, resulting in increased flow across the wall defect (which is detected by the embedded biosensors), along with changes in intraluminal blood flow characteristics in proximity to the graft defect. The ability of these embedded biosensors to record flow volume, directionality, and pressure in the database 113, 114, would provide compelling evidence of loss of device integrity and functionality. Furthermore, the ability to plot these flow related changes over time can demonstrate the speed at which device integrity is being compromised and the ensuing clinical risk to the patient if left unattended to.

Flow directionality is another important feature which can be assessed through the biosensors embedded within the device walls. In the case of an intravascular catheter, all flow would be expected to be antegrade (i.e., forward moving) and exhibit laminar flow characteristics. In the event that these flow characteristics are observed to change over time (e.g., to and fro, flow reversal, flow turbulence), one can begin to see evidence of loss of device functionality. This could be the result of early clot formation (e.g., formation of blood clot along the inner catheter wall) or external catheter obstruction (e.g., catheter tip obstructed by the blood vessel wall). In normal use cases, subtle and early changes related to intra-catheter flow would largely go unnoticed. However, by embedding biosensors within both the inner and outer walls of the device, these early functional changes in blood flow can be detected, analyzed by the program 110, and result in early intervention before a clinic adverse event (e.g., pulmonary embolism) occurs.

Functionality of a medical device can often be affected even when the device in question is in gross anatomic positioning based upon conventional surveillance techniques. As an example, if an ultrasound is performed to assess positioning of an intrauterine device (IUD), it is considered to be in satisfactory positioning when located within the endometrial cavity of the uterus. Along the same lines, a percutaneous gastrostomy tube visualized by radiography is considered to be in correct positioning when the gastrostomy tube balloon is situated in the gastric lumen. Over time, it is not unusual for either of these devices to undergo relatively minor changes in positioning which are not thought to be of clinical significance based upon these conventional medical imaging techniques. The IUD may be lodged up against the uterine wall (i.e., myometrium) which could both affect functionality and lead to a complication such as uterine wall perforation. The gastrostomy tube may similarly become wedged against the gastric wall which restricts free flow of tube injections or slip outside of the gastric lumen which can cause injected fluid to leak out into the peritoneal cavity or subcutaneous soft tissues. In most cases, these device positional changes will likely go undetected until a medical complication ensues, causing patient morbidity and potentially mortality.

However, with the present invention, embedded biosensors in the medical device can detect a number of ‘early signs” of positional change and functionality which can be analyzed by the program 110 and lead to early intervention and avoidance of morbidity. In the case of the IUD which has migrated along the myometrium, the biosensors can detect cellular tissue changes along the device walls (i.e., endometrium versus myometrium), which can signal the need to reposition the IUD. In the case of the migrating gastrostomy tube, the ability of embedded sensors in the gastrostomy tube walls and tip allows for program 110 simultaneous assessment of positioning and functionality (i.e., flow of fluid through the gastrostomy tube lumen). When functionality is compromised in any way, the sensor derived data will allow the program 110 to alert the clinical provider of the change, and prompt further investigation and/or clinical action. Because these sensor-derived measures are continuously collected and recorded over time, relatively small temporal changes will serve as triggers for the program 110 notification and intervention pathways.

As data is collected in the database 113, 114, best practice guidelines can be established and iteratively refined in accordance with longitudinal device positional data and community practice standards, in step 330 (see FIG. 3C). Pre-defined device positional data thresholds can be integrated into the database 113, 114 by the program 110, so as to provide a method for automated data analysis and communication to the clinical provider, along with an escalated notification pathway. If a predefined threshold is exceeded, an automated notification pathway can be triggered by the program 110, which alerts the designated clinical provider of the device optional change over time, the magnitude of the change, and the defined clinical urgency (based upon established practice standards). The clinical provider would in turn be required to acknowledge both receipt and understanding of the data. All data communications would be time stamped by the program 110, along with the identities of the sending and receiving parties. Any subsequent clinical actions taken would be recorded by the program 110 in the database 113, 114 for clinical outcomes analysis. In the event that the designated clinical provider fails to acknowledge receipt of the data or fails to act within a predefined period of time, the program 110 notification pathway would be escalated to alert the next designated provider in the notification pathway (e.g., department chief, historical administrator) to ensure prompt clinical action is taken.

Decision support can also be integrated by the program 110 to assist clinical providers with intervention options. By recording follow up data associated with the automated notification pathway, along with established clinical practice guidelines, decision support tools can be created by the program 110 to assist clinical providers with analysis of the device positional data and intervention options available. Customizable Decision Support can be undertaken in accordance with the specific medical device, patient profile, and provider profile.

A number of post-procedural actions can be taken in evaluation of device positioning and functionality, which can be classified as either routine or non-routine. Routine actions may include regularly scheduled diagnostic imaging or medical tests to confirm proper device position and functioning (i.e., device surveillance). Non-routine actions are diagnostic tests or clinical interventions which result from a specific concern related to device position and/or functionality. An example of a routine action may consist of a medical imaging exam performed at a routine interval to ensure proper device placement (e.g., daily chest radiograph to review placement of endotracheal tube).

The present invention includes a number of unique applications and tools which collectively serve to create a comprehensive system of quantitative accountability relating to medical devices and their clinical applications. In addition to the device itself, the quantitative accountability created by the program 110 using its derived database 113, 114, is applied to all contributing players including (but not limited to) the device manufacturer, vendor, institutional provider, individual clinical providers, patient, regulator, and payer. These players are continuously monitored and analyzed by the program 110 to ensure that their everyday work is compliant with community wide standards and best practice guidelines related to medical device use, beginning at the first step of device selection and ending with the last step of device disposal.

Unlike conventional data mining strategies, the present invention actually produces standardized and objective data which is used by the program 110 to create a database 113, 114 whose purpose is designed to provide meta-analysis of medical device safety, security, quality, and cost efficiency, and which collectively creates the opportunity for outcomes analysis. These derived analytics are designed to be customizable in relationship to a number of confounding variables including (but not limited to) the specific device, clinical provider, patient, clinical indication for device use, payer, and institution. The ability of the program 110 to sort data based upon these confounding variables provides for customized decision support and outcomes analysis which is both context and user specific. At the same time, the ability to collect and analyze this prospective data in real time, provides for an automated system of alerts and prompts when data outliers are identified which fall outside predefined levels of safety and quality.

The numerous components and applications contained within the invention include (but are not limited) medical device electronic tags and identification markers, tampering resistant and verifiable packaging, device embedded biomarkers and biosensors (for continuous data analysis), real-time device positional analysis, device quality control and quality assurance, real time assessment of device functionality, decision support, automated billing, and device disposal and recycling.

The creation of any one or all of these medical device components and applications can ultimately result in the creation of “smart medical devices”, which utilize the program 110 to provide real time data analytics and feedback specific to the individual device, clinical application, and end-user. The ultimate goal is to create a strategy for proactive intervention at the point of care, for the combined purposes of improved medical device safety, quality, and associated clinical outcomes.

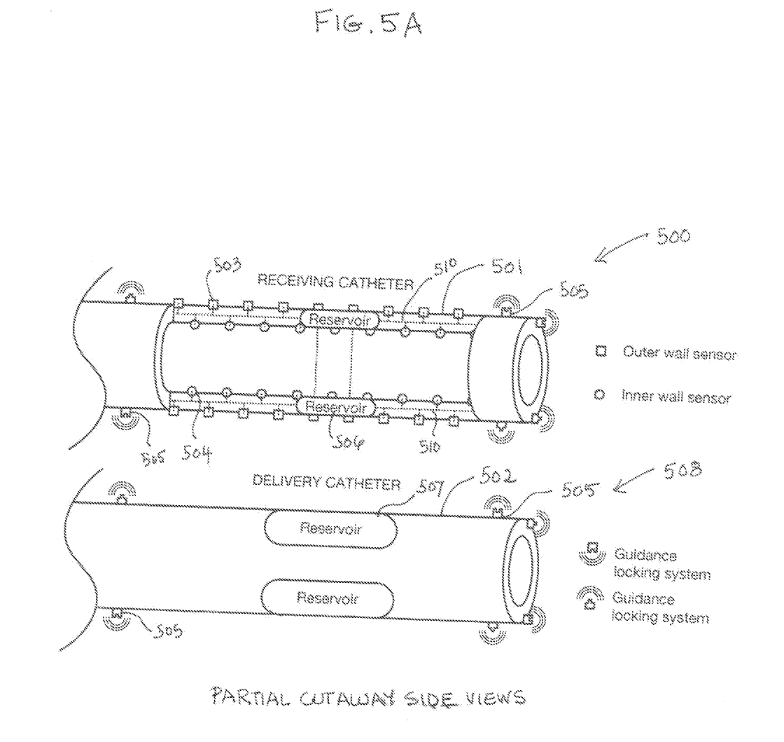

In one embodiment, a catheter-type medical device 500 of the present invention, includes a reservoir 506 for storage (see FIG. 5). In one embodiment, the sensor 503 embedded receiving catheter 501 contains storage reservoirs 506 which have interconnecting distribution channels 510 to individual biosensors outer wall sensors 503/inner wall sensors 504 within the catheter 501 wall. This provides for each individual biosensor 503/504 to function in an independent fashion from neighboring biosensors 503/504 when it comes to drug delivery or specimen sampling.

In the circumstance where one catheter (i.e., deliver catheter 502) is being used to deliver chemicals or drugs to a second catheter (i.e., receiving catheter 501), the reservoirs 507 from the delivery catheter 502 transfer internal contents to the reservoirs 506 of the receiving catheter 501 in a manner analogous to “in flight” airplane refueling. In this pattern of use, an embedded sensor guiding system 505 provides assistance in receiving device 500 localization by emitting an external “homing” signal (e.g., electronic signal, sound, light etc.) which can in turn help guide the delivery catheter 502. Once the delivery catheter 502 approaches the receiving catheter 501, external guiding mechanisms 505 in each of the catheters 501, 502 provide for physical alignment of the two catheters 501, 502 and their embedded sensors 503.

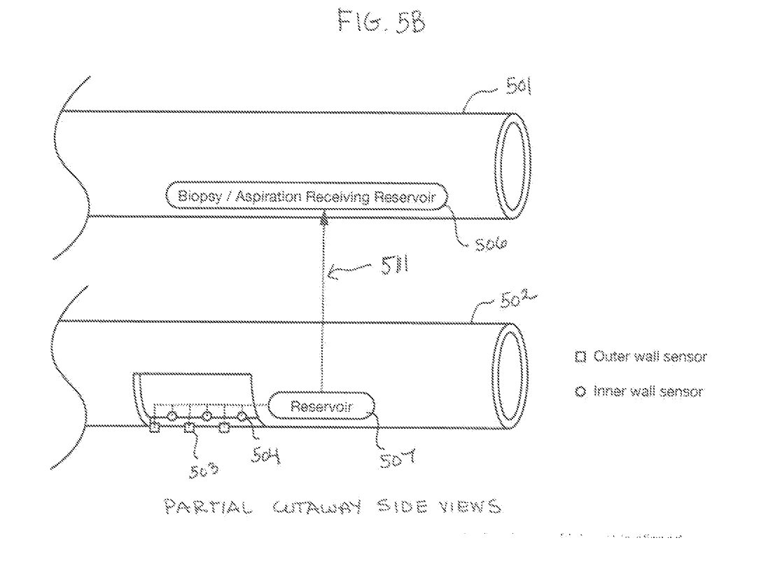

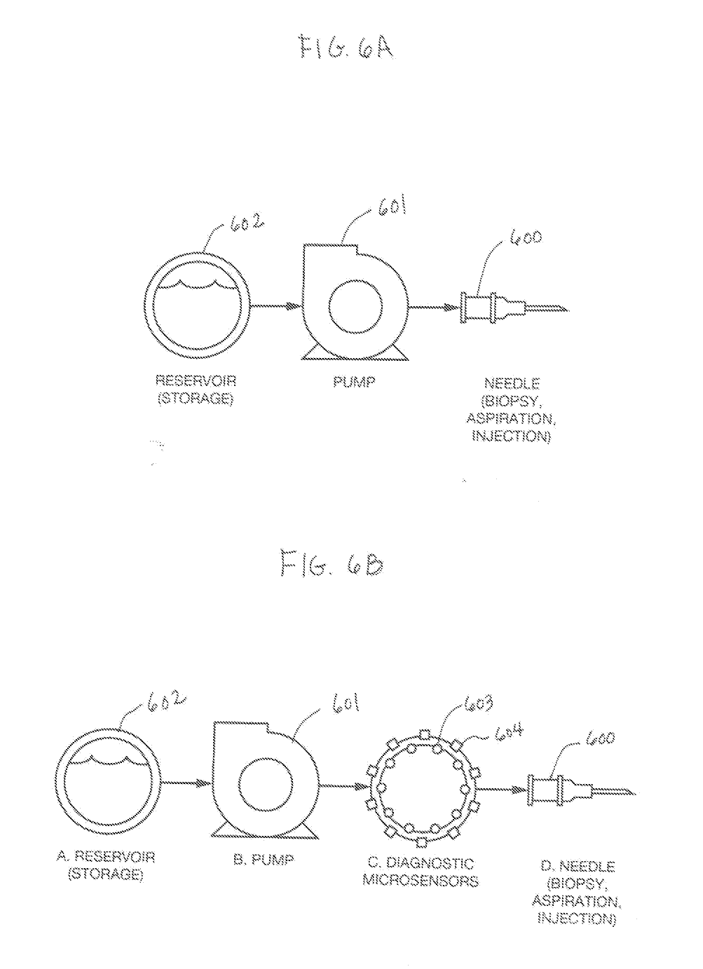

In one embodiment, once the receiving 501 and delivery 502 catheters 500 have been properly aligned, the injection apparatus of the delivery catheter 502 is engaged (see FIGS. 6A and 6B, for example). In this step, a needle 511 (see FIG. 5B, for example) which is attached to the reservoir 507 of the delivery catheter 502, is discharged, and in turn enters the receiving catheter 501 reservoir 506. Once these two reservoirs 506, 507 are connected (via the delivery catheter 502 needle), the contents of the delivery catheter 502 can be emptied into the receiving catheter 501 reservoir 506. For security purposes, a digital authentication code may be required prior to the emptying of the delivery catheter 502 reservoir 507. Once the transfer of reservoir 507 contents has been completed, the receiving catheter 501 reservoir(s) 506 can in turn transfer contents to individual sensor reservoirs 506 via the internal distribution channels 510 contained within the catheter 501 infrastructure.

At the time of targeted drug delivery, an available option is to engage dilatable balloons from each end of the catheter 501 thereby providing stasis of flow and allowing the delivered drug to remain in a relatively fixed sensor location (i.e., specific area of interest). The ability to have multiple reservoirs within an individual device provides for storage and distribution of multiple different drugs or chemical compounds used for different pathologies (e.g., infection, thrombolysis, chemotherapy).

In one embodiment, when a medical device directed biopsy or aspiration is performed, the focal area of interest is aligned with an individual biosensor (or group of biosensors). In this process, the abnormal tissue or cellularity is detected through the release of chemical compounds (e.g., prostaglandins, cytokines) or DNA sampling. Before the actual biopsy apparatus 511 is activated, a data verification step may be required to ensure that the original analysis of the presence of local pathology is confirmed. Once confirmed, the biopsy process is activated, with the corresponding needle(s) 511 from associated biosensors being released into the pathologic region of interest and suction is applied (via the corresponding pump apparatus of FIGS. 6A and 6B) to transfer the pathology specimen to the corresponding sensor reservoir 507 (see FIG. 5B) for temporary storage.

In a manner analogous to drug delivery transfer from the reservoir of one device to the reservoir of another device, a similar process can be used to transfer the pathology specimen from the reservoir of the original medical device to the storage reservoir of a second receiving device, which is then externally retrieved and emptied. This provides for more elaborate testing of the biopsy/aspiration specimen. After pathologic diagnosis is fully established, the same device and biosensors can be used for therapeutic intervention, which can take a variety of forms (e.g., drug delivery, radiation, thermal ablation).

In one embodiment, the retractable biopsy aspiration (injection) device 600 (for tissue biopsy and/or fluid aspiration), is shown in FIGS. 6A and 6B, along with the pump 601, and a reservoir 602. The pump 601 provides energy for needle deployment and function, and the catheter includes microsensors 603, 604 for diagnosis. When the needle 600 is deployed for aspiration or biopsy, the specimen obtained is transferred to the reservoir 602 for short term storage. This specimen can in turn be expelled from the reservoir 602 through the needle 600 into the specimen collection device. Similarly, when the needle 600 is used for drug delivery, the chemical compound to be delivered is transferred from the reservoir 602 to the needle 600, where it is then discharged. This illustrates the two-directional flow capabilities of the reservoir and needle apparatus, which in turn provides power via the associated pump mechanism.

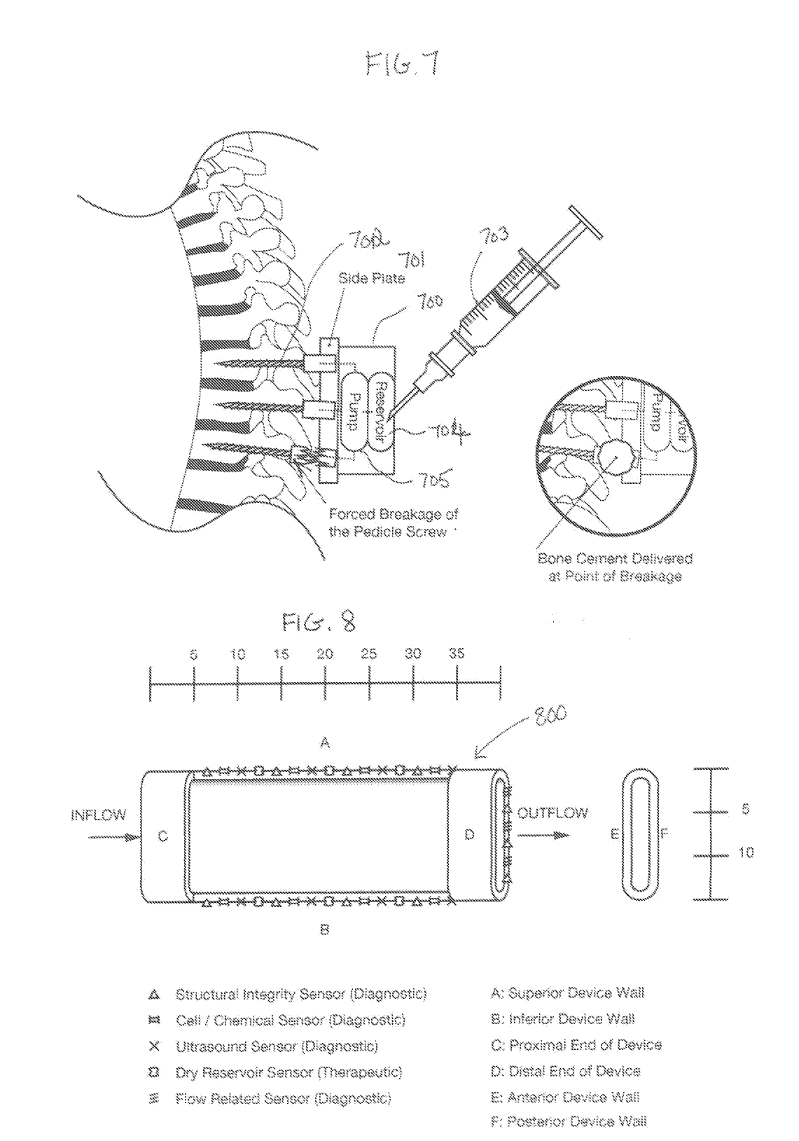

In one embodiment, a surgical device (see FIG. 7) 700 (e.g., spinal fixation hardware with side plate 701 and screws 702) is implanted for the treatment of underlying skeletal pathology (e.g., lumbar spine spondylolisthesis). During the course of routine biosensor analysis, a breakdown in device 700 integrity is detected, consisting of breakage of a pedicle screw 702, which can lead to pain and instability.

One strategy to conservatively manage the structural abnormality is to inject a mechanical stabilizer (e.g., methyl methacrylate) via a needle 703 in the specific region of structural deficiency. This can be accomplished by accessing the device reservoir 704 (which has been strategically located in a superficial location) under imaging guidance (e.g., CT, ultrasound), and introducing the chemical compound into the device reservoir 704 through a percutaneous injection (via needle 703). Once received in the reservoir 704, the pumping mechanism 705 of the device 700 can be deployed, which provides energy to deliver the chemical/drug to the specific sensors of interest, where it is injected into the adjacent tissues through a sensor deployed needle. The ability to continuously collect and analyze targeted biosensor derived data provides a unique method of measuring the success or failure of the specific intervention strategy and determining the need for additional and/or different intervention. The device database 110 in turn records all relevant data from the biosensors which can be used for creation of customizable best practice guidelines, technology assessment and refinement, and clinical outcomes analysis.

Device Specific Sensor Roadmap

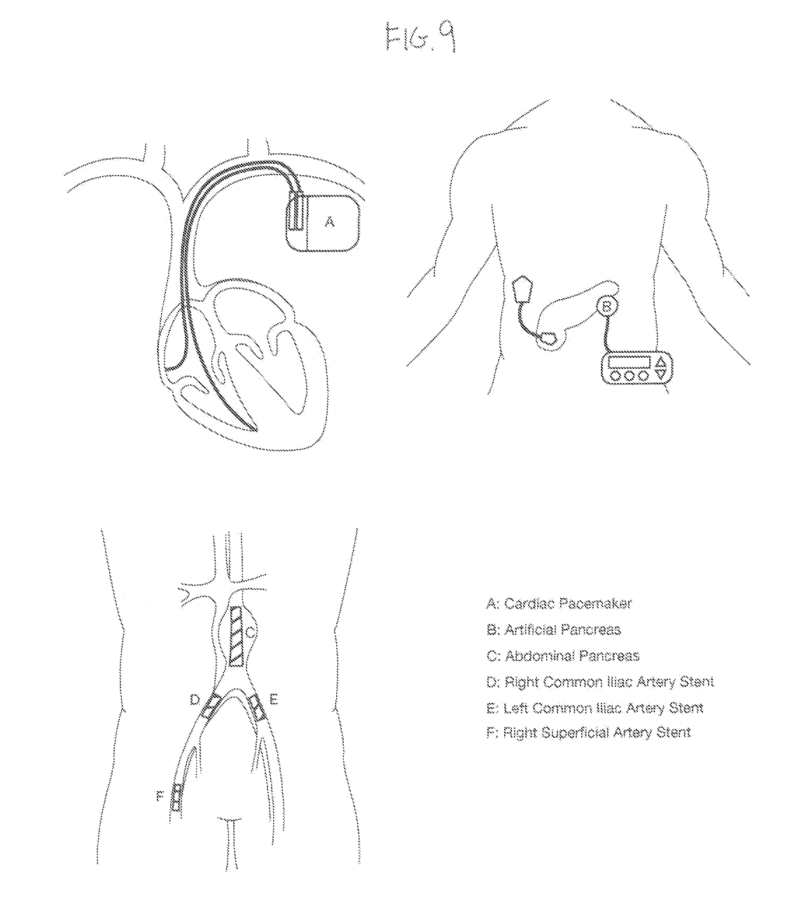

In one embodiment, the number, location, and functionality of individual biosensors within each individual medical device can be visualized in the form of a device specific sensor roadmap (see FIG. 8). This can be presented in a standardized format by the device manufacturer with electronic links of individual sensor specific data to the central medical device database 110. In the illustrated example of a vascular catheter 800, five different types of biosensors are contained within the device; four of which are diagnostic (i.e., structural integrity, flow characteristics, and detection of local cells/chemicals) and one of which is therapeutic (i.e., drug storage and release). In addition to identifying individual biosensors based upon their type and functionality, each individual biosensor can be localized on the basis of a standardized numerical wall distribution grid. Knowledge of individual sensor location can assist in a variety of functions including detection of focal pathology, device structural defects, sensor quality control (i.e., detection of individually malfunctioning sensors), and localized guidance of therapy.

In one embodiment, the Medical Device Data Components and Derived Analytics include responses to the following:

1. Clinical indication

Has requisite clinical data been properly documented in the patient medical record for the purposes of substantiating the medical procedure of record?

2. Medical procedure

Is the medical procedure consistent with established medical practice guidelines?

3. Patient Profile

What is the corresponding patient-specific risk factor associated with the procedure to be performed (i.e. risk/benefit analysis)?

Are alternative treatment and/or procedural options associated with an improved risk/benefit analysis for the clinical indication and patient profile?

4. Clinical providers charged with medical procedure decision making.

Were these individuals properly credentialed and authorized?

5. Clinical provider charged with the specific medical device selection.

Did this provider have all of the requisite education/training for optimal device selection?

Was there any perceived or inherent conflict of interest on the part of the provider in medical device selection?

6. Decision support resources used for procedure and device decision making

Was decision support technology and/or resources readily available to the clinical provider to assist in procedure/device selection? If so, what were they and how were they utilized?

7. Medical Device Database

Was the medical device database accessed, queried, and/or reviewed prior to procedure and device selection?

What specific database 113, 114 components were used in decision making (e.g., patient profile, device performance metrics, device cost/benefit analysis)?

8. Informed Consent

Was the requisite informed consent document satisfactorily obtained and recorded in the patient medical record?

Were alternative treatment and/or procedural options discussed with the patient (and/or designated decision maker) along with supporting medical device database statistics?

Was comparative institutional and individual provider performance data incorporated into the Patient Informed Consent in order to provide alternative provider options?

9. Device Security and Authentication

Was device identification and security data accurate and recorded in the database 113, 114?

10. Clinical providers involved in medical procedure performance.

Did these providers have the appropriate clinical/technical skill set, education/training, and credentials to perform the procedure?

What are the historical performance metrics associated with each individual provider in the specific procedure performed?

Identifying information of the institutional provider of the medical procedure.

What are the historical performance metrics associated with the institutional provider for the specific procedure performed?

9. Patient Medical Records

Were patient medical records reviewed (and documented in the database 113, 114) by the clinical provider/s prior to performance of the procedure?

Based upon this review of the patient medical records was there any modification of the planned procedure or device?

10. Procedural and Device Risk/Benefit Analysis

Was a patient, procedure, and device risk/benefit analysis performed based upon available patient and database 113, 114 data?

Were the planned procedure and device determined to be the optimal choice based on these analyses? If not, document why an alternative choice was made.

11. Procedural and Device Economic Analysis

Was the database 113, 114 used to determine the overall relative cost efficacy of the device selected for use? Were any patient specific economic variables (e.g., insurance coverage) used in device section?

How did the selected device compare with alternative options based on overall cost and clinical analysis?

12. Date and time of procedure performance.

Were there any delays or postponements of the planned procedure?

13. Duration of procedure performance.

How does this correspond to statistical analysis of comparable procedures (i.e., patient profile, institutional profile, clinical indication)?

14. Unexpected procedural delays.

Were there any events during the course of the procedure which caused an unexpected prolongation of the procedure performance time?

If yes, were these events related to patient (e.g., change in clinical status), provider (e.g., competing event causing delay or temporary stoppage), equipment (e.g., equipment breakdown) or device (e.g., breakage, malfunction)

15. Adverse events.

Did any adverse clinical event take place during or immediately following the performance of the procedure? If yes, document the specific type of event and its clinical severity.

How does the adverse event relate to statistical analysis of comparable procedures?

16. Follow-up care.

What specific actions were taken to ensure patient safety and optimize clinical outcomes of the procedure and device immediately following completion of the procedure?

Were these follow up actions (or lack thereof) consistent with established best practice guidelines?

17. Device Positioning

Was objective documentation of device positioning performed? If so, what was the standardized measure of device positioning (see above for measures)?

Was any subsequent action required for optimization of device positioning?

18. Functionality

Did the device performed as planned in accordance with the procedure and clinical profile?

If not, what commensurate action was taken?

How does the recorded degree of functionality compare with comparable procedures and patient profiles?

19. Economics

What is the comparative cost/benefit analysis for the device relative to competing devices used for similar clinical applications?

How does the device specific cost/benefit analysis change in accordance with specific clinical applications and patient profiles?

20. Device Longevity

What is the relative lifespan of the device for the specific (i.e., each individual clinical application) clinical use?

When devices are removed and/or replaced, what is the specific clinical and/or technical reason for removal/replacement?

21. Legal Ramifications

What is the relative frequency and associated cost of malpractice litigation associated with the medical device?

Are these legal ramifications related to individual Patient of Profile Profiles?

22. Regulatory Review

Has the device been associated with any warnings or other formal notices related to its use from regulatory (e.g., FDA) or professional societal organizations (e.g., AMA)?

If so, how has this restricted the clinical use of the device in question?

23. Utilization

What are the device usage patterns on local, regional, and national levels?

How are these usage patterns related to Patient, Provider, and Institutional

Profiles?

In the instance of statistical overuse, identify specific institutional or individual providers and any perceived or proven conflict of interest.

24. Device Refinement

Since its regulatory approval and initiation of clinical use, has the device in question undergone any technical revisions and/or refinements?

If so, how have these modifications affected overall device performance metrics?

25. Device Disposal

Was the device properly disposed of after its intended use was completed and was this disposal data documented in the database 113, 114?

Stepwise Medical Device Data Documentation (Medical Device Database)

1. Identification of Procedure

2. Medical Device Selection

3. Billing Authorization

4. Patient Registration

5. Provider/s Registration

6. Device Registration

7. Device Procurement and Security Mobilization

8. Patient Informed Consent

9. Procedural Preparation

10. Performance of Procedure

11. Post-Procedural Care

12. Device Positional Verification

13. Device Quality Control

14. Device Activation

15. Completion of Device and Procedural Data Documentation

16. Automated Billing

17. Post-Procedural Surveillance and Data Monitoring

18. Real-Time Device Data Analytics

19. Device Intervention (if applicable)

20. Device Disposal

It should be emphasized that the above-described embodiments of the invention are merely possible examples of implementations set forth for a clear understanding of the principles of the invention. Variations and modifications may be made to the above-described embodiments of the invention without departing from the spirit and principles of the invention. All such modifications and variations are intended to be included herein within the scope of the invention and protected by the following claims.

What is claimed is:

1. A computer-implemented method of providing ensuring medical device position and functionality, comprising:
providing a medical device for internal use within a patient during a medical procedure, said medical device having sensors or biomarkers disposed therein for providing data on said medical device and said patient;
confirming said medical device data integrity and device functionality by receiving said data from said medical device into a database of a computer system and performing an analysis of said data using a processor of said computer system; and
confirming, using said processor, a position of said medical device within said patient using an imaging device or a positional analysis of positional data from said data from said medical device;
wherein predetermined changes in said position of said medical device are monitored for indication of an adverse event.

2. The method of claim 1, wherein said medical device includes electronic tags which contain medical device information that can be scanned by a scanner and saved in said database.

3. The method of claim 2, wherein when a data outlier is detected during said analysis, performing a data reconciliation process using said processor, to identify erroneous, insufficient or abnormal data relative to best practice guidelines.

4. The method of claim 3, wherein when said data outlier is determined as abnormal, using said processor, generating an escalation pathway to analyze a cause and a severity of said data, in order to determine whether an intervention should be performed.

5. The method of claim 4, further comprising:
generating an alert by electronic means when a contraindication is identified during said analysis by said processor.

6. The method of claim 5, wherein said sensors or biomarkers provide continuous data after completion of said medical procedure.

7. The method of claim 6, wherein an appropriateness of said medical procedure and said medical device are included in said analysis.

8. The method of claim 7, wherein a standardized model for training, education, and proof of clinical competency with respect to medical devices is determined during said analysis.

9. The method of claim 6, wherein a GPS in said medical device provides anatomic real-time position and continuous data.

10. The method of claim 9, comparing data on said position of said medical device within said patient with comparable patients and medical devices using said processor.

11. The method of claim 10, wherein said analysis includes clinical outcomes analysis and analysis of providers to generate customized medical device decision-making relative to peer and community wide standards.

12. The method of claim 11, further comprising:
continuously monitoring quality and safety metrics of at least patients, providers, and said medical devices.

13. The method of claim 12, further comprising:
generating best practices guidelines using said processor, based on said compared data, for use of said medical device with patients
